(19) United States
(12) Patent Application Publication
(10) Pub. No.: US 2020/0078295 A1
(43) Pub. Date: Mar. 12, 2020

(54) Low Dose Doxepin Formulations, Including Buccal, Sublingual And Fastmelt Formulations, And Uses Of The Same To Treat Insomnia

(71) Applicant: Currax Pharmaceuticals LLC, Morristown, NJ (US)

(72) Inventors: Luigi Schioppi, Escondido, CA (US); Brian T. Dorsey, Encinitas, CA (US); Michael Skinner, San Diego, CA (US); John Carter, Keswick (CA); Terry Cobb, Steamboat Springs, CO (US); Neil B. Kavey, Chappaqua, NY (US)

(21) Appl. No.: 16/686,113

(22) Filed: Nov. 16, 2019

Related U.S. Application Data
(63) Continuation of application No. 12/301,223, filed on Jul. 14, 2011.
(60) Provisional application No. 60/833,319, filed on Jul. 25, 2006.
(60) Provisional application No. 60/801,823, filed on May 19, 2006.
(60) Provisional application No. 60/801,824, filed on May 19, 2006.

(51) Int. Cl. A61K 9/00; A61K 31/335

(52) U.S. Cl. CPC A61K 9/0056; A61K 31/335

(57) ABSTRACT

The invention disclosed herein generally relates to low-dose oral doxepin pharmaceutical formulations and the use of these formulations to promote sleep.

### FIELD OF THE INVENTION

Embodiments of the invention disclosed herein relate to low-dose, oral, transmucosal doxepin pharmaceutical formulations, including buccal, sublingual, and fastmelt formulations, methods of making the formulations, and the use of these formulations to promote sleep.

### BACKGROUND OF THE INVENTION

Low doses of doxepin can be used to treat sleep disorders, such as insomnia. Sleep is essential for health and quality of life. Insomnia is a growing health problem in the United States. It is believed that more than 10-15 million people suffer from chronic insomnia and up to an additional 70 million people suffer from some form of insomnia each year. Insomnia is a condition characterized by difficulty falling asleep (sleep onset), waking frequently during the night (fragmented sleep), waking too early (premature final awakening), and/or waking up feeling un-refreshed. In the National Sleep Foundation's (NSF) Sleep in America Poll 2005, 42% of survey respondents reported that they awoke frequently during the night, 22% of adults reported waking too early and not being able to return to sleep and 38% reported waking and feeling un-refreshed.

There are numerous limitations of the currently available hypnotic drugs and there is no one drug that can address all three components of insomnia efficacy (onset, maintenance and duration) with a clean safety profile. Side effects of hypnotics include: residual sedation, lethargy, disorientation, impaired psychomotor function including light headedness and falls, amnesia, headaches, agitation, nightmares, dry mouth, metallic taste, complex sleep behaviors, dependence, withdrawal syndrome and rebound insomnia. The side effects are often worse in elderly patients. These drugs also may be subject to the additional problems of addictive potential and tolerance. The limitations of the currently available hypnotics are reflected in current medical guidelines that recommend and emphasize the use of non drug treatment options as first line therapy.

Embodiments of the invention provide low-dose, oral, transmucosal formulations of doxepin and doxepin compounds, and address the unique aspects of sleep disorders.

### SUMMARY OF THE INVENTION

Embodiments of the invention disclosed herein relate to low-dose doxepin formulations, and methods of making and using the same. Preferably, some embodiments relate to oral, transmucosal formulations. Transmucosal delivery offers the potential for once daily dosing of oral drugs and can avoid, at least in part, the effects of first pass metabolism.

As discussed more fully below, many of the pharmacokinetics of low-dose doxepin, in particular for use in treating sleep disorders, have not been known or fully appreciated. As one example, although doxepin dissolves quickly in the stomach, it undergoes a significant first pass extraction or metabolism in the liver. This first pass extraction can influence the impact of the therapeutic effect of the administered doxepin. For example, it can result in a delay in the onset of the sleep promoting action of the drug. Thus, some embodiments relate to formulations that can be administered so as to be absorbed or taken up, at least in part, in the mouth and/or intestines.

In view of the previously unrecognized characteristics of doxepin when used to treat sleep, it can be desirable to achieve any of several different objectives when using transmucosal pharmaceutical dosage forms. For example, preferably the dosage form can be uniform with respect to drug substance content, fast dissolving, stable, palatable, and otherwise acceptable to patients in order to maximize patient compliance. In certain contexts, early and/or accelerated onset of drug action also can be advantageous. For example, in the context of sleep, early onset of drug action can be important due to the discreet window of time in which a patient needs to sleep. Also in the context of sleep, the dosage form preferably maintains sleep for a full 7 or 8 hour sleep cycle without significant next-day sedation.

The selected dosage level of a drug to be administered to a patient can depend upon, for example, the route of administration, the severity of the condition being treated, and the condition and prior medical history of the patient being treated. It will be understood, however, that the specific dose level for any particular patient can depend upon a variety of factors including the genetic makeup, body weight, general health, diet, time and route of administration, combination with other drugs and the particular condition being treated, and its severity. For the treatment of insomnia, preferably one dose is administered prior to bedtime.

The selected dosage can also be determined by targeting a mean plasma concentration profile that has been associated with improvement in one or more polysomnography sleep variables including latency to persistent sleep, wake after sleep onset, total sleep time, sleep efficiency, wake time during sleep, or wake time after sleep. Due to the limited time frame for the treatment of insomnia, drug formulations that achieve an effective plasma concentration are desirable. The clinically effective drug plasma concentration and duration of said drug plasma concentration may be achieved by any suitable route of administration including oral, buccal, sublingual, transmucosal, or intestinal using any suitable formulation.

The issue of intestinal absorption and first pass metabolism can make it difficult to achieve effective levels of drug in the plasma. In order to by pass issues of intestinal absorption and first pass drug metabolism, drug dosage forms that are oral disintegrating and absorbed through the oral mucosa can be used.

In the treatment of insomnia it can be desirable to have a blood plasma concentration of drug in a therapeutically effective range as rapid as possible for example, to induce sleep onset as quickly as possible. Additionally, it can be desirable to have the dose of drug in the plasma be maintained through the sleep period to maintain the beneficial effects on other sleep parameters, such as awakenings after sleep onset and sleep duration.

In some embodiments, a patient with insomnia, can be treated with an orally disintegrating formulation of doxepin, pharmaceutically acceptable salts, or other doxepin-related compounds that can be partially or substantially absorbed in the oral mucosa to afford a therapeutic blood plasma level that can be maintained through the sleep period. In some embodiments, the orally disintegrating formulation of doxepin, pharmaceutically acceptable salts, or other doxepin-related compounds when taken by a patient can afford a therapeutically effective blood plasma concentration within 60 minutes and be maintained at a therapeutically relevant level throughout the sleep period. In some embodiments, the orally disintegrating formulation of doxepin, pharmaceutically acceptable salts, or other doxepin-related compounds when taken by a patient can afford a therapeutically effective blood plasma concentration within 40 minutes and be maintained at a therapeutically relevant level throughout the sleep period. In some embodiments, the orally disintegrating formulation of doxepin, pharmaceutically acceptable salts, or other doxepin-related compounds when taken by a patient can afford a therapeutically effective blood plasma concentration of doxepin within 20 minutes and be maintained at a therapeutically relevant level throughout the sleep period. In some aspects, the doxepin, pharmaceutically acceptable salts, or other doxepin-related compounds that is absorbed through the oral mucosa can afford a rapid elevation of drug in the blood plasma to above the therapeutic level that lessens, although still being maintained above the therapeutic level, and then slowly rising as additional drug is absorbed intestinally. In some aspects, substantially all of the doxepin can be absorbed through the oral mucosa affording a rapid elevation of drug in the blood plasma to above the therapeutic level that is maintained above the therapeutic level throughout the sleep period.

Also, some embodiments relate to manufacturing processes for the formulations, as well as methods of using the formulations. In some aspects the formulations have one or more desirable physical properties, have preferable functional characteristics, and/or permit efficient and economical manufacturing of low-dose, oral, transmucosal doxepin dosage forms.

It may be desirable to have a manufacturing process that is economical, efficient, robust, and preferably, simple-requiring a minimal number of steps and/or excipients. Furthermore, the active ingredient and excipients preferably have suitable flow properties to ensure efficient mixing and acceptable content uniformity, weight uniformity, potency, disintegration, dissolution, hardness, and friability of the final dosage form. Good flow properties also may be beneficial for precise volumetric feeding of the material to a die cavity. However, efficient mixing and acceptable content uniformity are difficult to achieve for low dose dosage forms.

Mixed particle sized powders can segregate due to operational vibrations and/or fluidization resulting in final dosage forms with poor drug or active pharmaceutical ingredient (API) content uniformity. Active substances with a small particle size mixed with excipients having a larger particle size will typically segregate or de-mix during the formulation process. The problem of small particle size and poor flowability can be addressed by enlarging the particle size of the active substance, usually by granulation of the active ingredient either alone or in combination with a filler and/or other conventional excipients.

Accordingly, embodiments of the invention disclosed herein address and achieve one or more of the above-mentioned considerations. Some embodiments surprisingly achieve several or many of the considerations.

In particular, embodiments disclosed herein relate to oral, transmucosal dosage forms comprising low doses of doxepin hydrochloride, methods of manufacturing these dosage forms, and methods of using the formulations and dosage forms. Preferably, the low doses of doxepin hydrochloride can be provided as rapidly disintegrating dosage forms, as described herein, that can be advantageously used for treatment of insomnia. In some aspects, the formulations have one or more of improved friability, compression, dissolution, uniformity, disintegration, palatability, and the like. Also, in some aspects, the formulations can permit at least one or more of rapid onset, greater and/or more rapid plasma levels, and the like. In some aspects the formulations can result in more rapid drug action (e.g., sleep onset) without otherwise significantly changing the pharmacokinetic profile of low dose doxepin seen with other formulations.

Some embodiments provide a method for treating insomnia comprising administering to a patient an oral-mucosal low dose of doxepin, a pharmaceutically acceptable salt thereof, or doxepin-related compounds. The administered substance can be delivered, for example, in a daily dosage ranging from about 0.01 to about 10 milligrams. In some embodiments, the daily dosage can range, for example, from about 0.5 to about 9 milligrams, from about 1 to about 9 milligrams, from about 1 to about 6 milligrams, from about 1 to about 3 milligrams, from about 2 to about 3 milligrams, or the like. The pharmaceutically acceptable salt can be any salt, including for example the hydrochloride salt. Also, the prodrug can be any prodrug.

Some embodiments relate to treating insomnia comprising administering to a patient doxepin, a pharmaceutically acceptable salt thereof, or doxepin-related compounds, in an orally disintegrating dosage form that can be substantially absorbed across the oral mucosa to avoid, at least in part, intestinal absorption and first pass metabolism. For example, in some embodiments, between about 1% and 99%, or between about 5% and 95%, or between about 10% and 90% of the doxepin, or pharmaceutically acceptable salts, or other doxepin-related compounds, can be absorbed across the oral mucosa thereby avoiding intestinal absorption and first pass metabolism. Also, in some embodiments, greater than 80% of the doxepin, or pharmaceutically acceptable salts, or other doxepin-related compounds, thereof can be absorbed across the oral mucosa thereby avoiding intestinal absorption and first pass metabolism. In some embodiments, greater than 60% of the doxepin, or pharmaceutically acceptable salts, or other doxepin-related compounds, can be absorbed across the oral mucosa thereby avoiding intestinal absorption and first pass metabolism. In some embodiments, greater than 40% of the doxepin, or pharmaceutically acceptable salts thereof, or other doxepin-related compounds, can be absorbed across the oral mucosa thereby avoiding intestinal absorption and first pass metabolism. In some embodiments, greater than 20% of the doxepin, or pharmaceutically acceptable salts thereof, or other doxepin-related compounds, can be absorbed across the oral mucosa thereby avoiding intestinal absorption and first pass metabolism. In some embodiments, the doxepin that is not absorbed across the oral mucosa can be absorbed intestinally following the normal pharmacokinetics for this drug.

Preferred embodiments provide a method for treating insomnia comprising administering to a patient doxepin, a pharmaceutically acceptable salt thereof, or doxepin-related compounds in an orally disintegrating formulation where the blood plasma level of doxepin reaches a therapeutic level within 60 minutes of administration. Further, in some embodiments, a therapeutically effective blood plasma level is achieved within 40 minutes of administration of doxepin, or a pharmaceutically acceptable salt thereof. In a preferred embodiment, a therapeutically effective blood plasma level is achieved within 20 minutes of administration of doxepin, or a pharmaceutically acceptable salt thereof.

Preferred embodiments provide methods of treating sleep onset insomnia, comprising administering to a patient doxepin, a pharmaceutically acceptable salt thereof, or doxepin-related compounds thereof in an orally disintegrating formulation where the blood plasma level of doxepin reaches a therapeutic level within 60 minutes of administration. Further, in some embodiments, a therapeutic blood plasma level of doxepin is achieved within 40 minutes of administration of doxepin, or a pharmaceutically acceptable salt thereof. In a preferred embodiment, a therapeutic blood plasma level of doxepin is achieved within 20 minutes of administration of doxepin, or a pharmaceutically acceptable salt thereof.

In some aspects, an orally disintegrating formulation of about 6 mg of doxepin, or pharmaceutically acceptable salts, or other doxepin-related compounds can be substantially dissolved within 5 minutes and a therapeutically effective plasma concentration of doxepin achieved within 60 minutes. “Substantially dissolved” as used herein can mean that approximately 1% to about 99%, 1% to about 75%, or 1% to about 50% of the formulation can be dissolved within the specified time frame. In some aspects, the orally disintegrating formulation can be substantially dissolved within 5 minutes and a therapeutically effective plasma concentration of doxepin achieved within 40 minutes. In some aspects, the orally disintegrating formulation can be substantially dissolved within 5 minutes and a therapeutically effective plasma concentration of doxepin achieved within 20 minutes.

In some aspects, an orally disintegrating formulation of about 3 mg of doxepin, pharmaceutically acceptable salts thereof, or other doxepin-related compounds can be substantially dissolved within 5 minutes and a therapeutically effective plasma concentration of doxepin achieved within 60 minutes. In some aspects, the orally disintegrating formulation can be substantially dissolved within 5 minutes and a therapeutically effective plasma concentration of doxepin achieved within 40 minutes. In some aspects, the orally disintegrating formulation can be substantially dissolved within 5 minutes and a therapeutically effective plasma concentration of doxepin achieved within 20 minutes.

In some aspects, an orally disintegrating formulation of about 1 mg of doxepin, pharmaceutically acceptable salts thereof, or other doxepin-related compounds can be substantially dissolved within 5 minutes and a therapeutically effective plasma concentration of doxepin achieved within 60 minutes. In some aspects, the orally disintegrating formulation can be substantially dissolved within 5 minutes and a therapeutically effective plasma concentration of doxepin achieved within 40 min. In some aspects, the orally disintegrating formulation can be substantially dissolved within 5 minutes and a therapeutically effective plasma concentration of doxepin achieved within 20 minutes.

In some aspects, an orally disintegrating formulation of about 6 mg of doxepin, a pharmaceutically acceptable salt thereof can be substantially disintegrated within 60 seconds and a therapeutically effective plasma concentration of doxepin achieved within 60 minutes. In some aspects, the orally disintegrating formulation can be substantially disintegrated within 60 seconds and a therapeutically effective plasma concentration of doxepin achieved within 40 minutes. In some aspects, the orally disintegrating formulation can be substantially disintegrated within 60 seconds and a therapeutically effective plasma concentration of doxepin achieved within 20 minutes.

In some aspects, an orally disintegrating formulation of 3 mg of doxepin, a pharmaceutically acceptable salt thereof can be substantially disintegrated within 60 seconds and a therapeutically effective plasma concentration of doxepin achieved within 60 minutes. In some aspects, the orally disintegrating formulation can be substantially disintegrated within 60 seconds and a therapeutically effective plasma concentration of doxepin achieved within 40 minutes. In some aspects, the orally disintegrating formulation can be substantially disintegrated within 60 seconds and a therapeutically effective plasma concentration of doxepin achieved within 20 minutes.

In some aspects, an orally disintegrating formulation of 1 mg of doxepin, a pharmaceutically acceptable salt thereof can be substantially disintegrated within 60 seconds and a therapeutically effective plasma concentration of doxepin achieved within 60 minutes. In some aspects, the orally disintegrating formulation can be substantially disintegrated within 60 seconds and a therapeutically effective plasma concentration of doxepin achieved within 40 minutes. In some aspects, the orally disintegrating formulation can be substantially disintegrated within 60 seconds and a therapeutically effective plasma concentration of doxepin achieved within 20 minutes.

In some aspects, the doxepin, or a pharmaceutically acceptable salt thereof can be formulated in a rapidly orally disintegrating dosage form so that between about 1% and 99%, or between about 5% and 95%, or between about 10% and 90% of the doxepin is absorbed across the oral mucosa. In some embodiments, the doxepin or a pharmaceutically acceptable salt thereof can be formulated in a rapidly orally disintegrating dosage form so that greater than 70% of the doxepin is absorbed across the oral mucosa. In some embodiments, the doxepin or a pharmaceutically acceptable salt thereof can be formulated in a rapidly orally disintegrating dosage form so that greater than 50% of the doxepin is absorbed across the oral mucosa. In some embodiments, the doxepin, or a pharmaceutically acceptable salt thereof can be formulated in a rapidly orally disintegrating dosage form so that greater than 30% of the doxepin is absorbed across the oral mucosa. In some embodiments, the doxepin, or a pharmaceutically acceptable salt thereof can be formulated in a rapidly orally disintegrating dosage form so that greater than 10% of the doxepin is absorbed across the oral mucosa.

In some aspects the insomnia can be a chronic insomnia or a non-chronic insomnia. For chronic (e.g., greater than 3-4 weeks) or non-chronic insomnias, a patient may suffer from difficulties in sleep onset, sleep maintenance (interruption of sleep during the night by periods of wakefulness), sleep duration, sleep efficiency, premature early-morning awakening, or a combination thereof. Also, the insomnia may be attributable to the concurrent use of other medication, for example. The non-chronic insomnia can be, for example, a short term insomnia or a transient insomnia. The chronic or non-chronic insomnia can be a primary insomnia or an insomnia that is secondary to another condition, for example a disease such as depression or chronic fatigue syndrome. In some aspects, the patient can be one that is not suffering from an insomnia that is a component of a disease, or a patient can be treated that is otherwise healthy. As previously mentioned, the chronic or non-chronic insomnia can be a primary insomnia, that is, one that is not attributable to another mental disorder, a general medical condition, or a substance. In many cases, such conditions may be associated with a chronic insomnia and can include, but are not limited to, insomnia attributable to a diagnosable DSM-IV disorder, a disorder such as anxiety or depression, or a disturbance of the physiological sleep-wake system. In some aspects the insomnia can be non-chronic, or of short duration (e.g., less than 3-4 weeks). Examples of causes of such insomnia may be extrinsic or intrinsic and include, but are not limited to environmental sleep disorders as defined by the International Classification of Sleep Disorders (ICSD) such as inadequate sleep hygiene, altitude insomnia or adjustment sleep disorder (e.g., bereavement). Also, short-term insomnia may also be caused by disturbances such as shift-work sleep disorder.

Also, some embodiments relate to a composition that includes doxepin, pharmaceutically acceptable salts thereof, and/or other doxepin-related compounds. In a preferred embodiment, such composition of doxepin or a pharmaceutically acceptable salt thereof can be provided in a daily dosage and/or unit dosage ranging from about 0.01 to about 10 milligrams.

The pharmaceutically acceptable salt of doxepin can be a hydrohalic acid salt. For example, the pharmaceutically acceptable salt of doxepin can be the hydrochloride salt.

Embodiments of the invention disclosed herein relate to buccal, sublingual and fastmelt low-dose doxepin formulations. Also, some embodiments relate to manufacturing processes for the formulations, as well as methods of using the formulations. In some aspects the formulations have one or more desirable physical properties, have preferable functional characteristics, and/or permit efficient and economical manufacturing of low dose doxepin dosage forms.

Making low dose formulations can present technical and economic challenges that are not present for higher dose formulations. Furthermore, currently marketed doxepin formulations do not take into account the unique aspects of sleep disorders.

Embodiments of the invention provide orally disintegrating low dose formulations of doxepin and doxepin compounds, and also addresses and overcomes the challenges and problems associated with formulating and manufacturing orally disintegrating low-dose doxepin dosage forms. Additionally, embodiments of the invention provide that doxepin, pharmaceutically acceptable salts thereof, or other doxepin-related compounds can be substantially absorbed across the oral mucosa to afford a therapeutically effective plasma concentration of doxepin concentration within 60 minutes of treatment.

Additional embodiments disclosed herein relate to methods of manufacture for low-dose dosage forms of doxepin, including, for example, on a large scale. In a preferred embodiment, the methods of manufacture can achieve uniformity of drug substance content and overcome segregation issues that can plague low dose formulations, and can do so in an economic and efficient manner. Some embodiments of the invention relate to low dose doxepin formulations that are amenable to direct compression and that produce a high yield of buccal, sublingual and/or fastmelt low-dose doxepin dosage forms having acceptable content uniformity, and hardness, and friability.

While it is not intended the invention disclosed herein be limited to any specific formulation, in a preferred embodiment, said fast disintegrating doxepin is an orally disintegrating tablet (ODT). In a preferred embodiment said ODT is formulated with doxepin HCl. It is not intended that the invention be limited to any specific dosage of doxepin in a given dosage form. However, in a preferred embodiment, the dosage of doxepin, per ODT, is in the range between 1 mg and 9 mg. Further, in a more preferred embodiment, the dosage of doxepin, per ODT, is 1 mg or 3 mg or 6 mg.

In other embodiments, the invention also relates to methods for the administration of fast disintegrating, oral, transmucosal formulations of doxepin which rapidly exert a therapeutic effect to treat sleep onset, for example, buccal, sub-lingual and ODT formulations. While it is not intended the invention be limited to any specific formulation, in a preferred embodiment, said fast disintegrating formulation is an ODT (Orally Disintegrating Tablet).

Some embodiments relate to a pharmaceutical composition that can comprise from about 0.1 to about 9 mg of doxepin, or a pharmaceutically acceptable salt, prodrug, or metabolite thereof, and from about 15% to about 99.9% w/w of at least one quick dissolve delivery system or at least one directly compressible excipient for orally disintegrating formulations. In some embodiments, the orally disintegrating composition substantially disintegrates and partially dissolves in the oral cavity.

In some embodiments, pharmaceutical composition that can comprise from about 0.1 to about 9 mg of doxepin, or a pharmaceutically acceptable salt, prodrug, or metabolite thereof, and from about 50% to about 90% w/w Pharmaburst, or other similar specialty excipient. Pharmaburst is a specialty excipient designed specifically for ODT applications supplied by SPI Pharma. The composition can further comprise doxepin, or a pharmaceutically acceptable salt, prodrug, or metabolite thereof in an amount of about 1 mg to about 9 mg. For example, doxepin, or a pharmaceutically acceptable salt, prodrug, or metabolite thereof can be provided in an amount of about 3 mg. In another embodiment, doxepin, or a pharmaceutically acceptable salt, prodrug, or metabolite thereof can be provided in an amount of about 6 mg. In a preferred embodiment, the pharmaceutical composition can contain from about 60% to about 85% w/w Pharmaburst. In a more preferred embodiment, the pharmaceutical composition can contain from about 70% to about 80% w/w Pharmaburst. In some embodiments, the amount of Pharmaburst in the pharmaceutical composition can be 50%, 60%, 70% 75%, 80%, 81%, 82%, 83%, 84%, 85% w/w. Thus, in one example, the pharmaceutical composition can contain Pharmaburst in an amount of about 83% w/w and doxepin, or a pharmaceutically acceptable salt, prodrug, or metabolite thereof in an amount of about 6 mg. In another embodiment, the pharmaceutical composition can contain Pharmaburst in an amount of about 83% w/w and doxepin, or a pharmaceutically acceptable salt, prodrug, or metabolite thereof in an amount of about 3 mg. In still another embodiment, the pharmaceutical composition can contain Pharmaburst in an amount of about 83% w/w and doxepin, or a pharmaceutically acceptable salt, prodrug, or metabolite thereof in an amount of about 1 mg. In some embodiments the pharmaceutical composition can be a tablet, a lozenge, a chewing gum, a film, a capsule, a gel cap, a caplet, a pellet, or a bead.

In some embodiments, the pharmaceutical compositions disclosed herein comprise from about 0.1 to about 9 mg of doxepin, or a pharmaceutically acceptable salt, prodrug, or metabolite thereof, and from about 15% to about 30% w/w RxCIPIENTS™ FM1000. RxCIPIENTS™ FM1000 is a specialty excipient designed specifically for ODT applications comprised of calcium silicate, supplied by Huber. In a preferred embodiment, the pharmaceutical compositions contain from about 18% to about 27% w/w RxCIPIENTS™ FM1000. In a more preferred embodiment, the pharmaceutical compositions contain from about 20% to about 25% w/w RxCIPIENTS™ FM1000. In one embodiment, the pharmaceutical composition can contain RxCIPIENTS™ FM1000 in an amount of about 20-30% w/w and doxepin, or a pharmaceutically acceptable salt, prodrug or metabolite thereof in an amount of about 1 mg. In another embodiment, the pharmaceutical composition can contain RxCIPIENTS™ FM1000 in an amount of about 20-30% w/w and doxepin, or a pharmaceutically acceptable salt, prodrug, or metabolite thereof, in an amount of about 6 mg. In still another embodiment, the pharmaceutical composition can contain RxCIPIENTS™ FM1000 in an amount of about 20-30% w/w and doxepin, or a pharmaceutically acceptable salt, prodrug, or metabolite thereof, in an amount of about 3 mg. In some embodiments, RxCIPIENTS™ FM1000 is used in combination with another excipient such as mannitol, microcrystalline cellulose, dicalcium phosphate, and the like. In some embodiments, the pharmaceutical composition can be a tablet, a lozenge, a chewing gum, a film, a capsule, a gel cap, a caplet, a pellet, or a bead.

In some embodiments, pharmaceutical compositions disclosed herein can comprise from about 0.1 to about 9 mg of doxepin, or a pharmaceutically acceptable salt, prodrug, or metabolite thereof, and from about 25% to about 60% w/w F-Melt™ Type M or F-Melt™ Type C. F-Melt™ is a specialty excipient designed specifically for ODT applications supplied by Fuji Chemicals of Japan. In a preferred embodiment, the pharmaceutical composition can contain from about 30% to about 55% w/w F-Melt™. In a more preferred embodiment, the pharmaceutical composition can contain from about 40% to about 50% w/w F Melt™. In one embodiment, the amount of F-Melt™ in the pharmaceutical composition can be about 58% w/w. Thus, in one embodiment, the pharmaceutical composition can contain F-Melt™ an amount of about 58% w/w and doxepin, or a pharmaceutically acceptable salt, prodrug, or metabolite thereof in an amount of about 6 mg. In another embodiment, the pharmaceutical composition can contain F-Melt™ in an amount of about 58% w/w and doxepin, or a pharmaceutically acceptable salt, prodrug, or metabolite thereof, in an amount of about 3 mg. In still another embodiment, the pharmaceutical composition can contain F-Melt™ in an amount of about 58% w/w and doxepin, or a pharmaceutically acceptable salt, prodrug, or metabolite thereof in an amount of about 1 mg. In some embodiments the pharmaceutical composition can be a tablet, a chewing gum, a lozenge, a film, a capsule, a gel cap, a caplet, a pellet, or a bead.

In some embodiments the composition can be in the form of a tablet, a lozenge, a chewing gum, a capsule, a gel cap, a caplet, a pellet, a bead, or the like, where the composition can have a total mass of about 50 mg to about 300 mg. In a preferred embodiment the composition can have a mass of from about 75 mg to about 100 mg. In some embodiments, the composition can have a total mass of 50 mg, 75 mg, 80 mg, 90 mg, 100 mg, 125 mg, 150 mg, 200 mg, 250 mg, or 300 mg.

In some embodiments, the pharmaceutical composition can comprise from about 0.1 to about 9 mg of doxepin, or a pharmaceutically acceptable salt, prodrug, or metabolite thereof, and at least one excipient. The excipient can be selected from, for example, but not limited to, RxCIPIENTS™ FM1000, Pharmaburst, F-Melt™, silicified microcrystalline cellulose, microcrystalline cellulose, lactose, a compressible sugar, xylitol, sorbitol, mannitol, such as Perlitol 200 SD, pregelatinized starch, maltodextrin, calcium phosphate dibasic, calcium phosphate tribasic, calcium carbonate DC, a calcium silicate, and the like.

In some embodiments, the pharmaceutical composition can be comprised from about 0.1 to about 9 mg of doxepin, or a pharmaceutically acceptable salt, prodrug, or metabolite thereof, and Perlitol 200 SD. In some aspects, the Perlitol 200 SD can be provided in amount of about 20% to about 85% w/w. In a preferred aspect, the Perlitol 200 SD can be provided in amount of about 15% to about 45% w/w. In a further preferred aspect, 45% to about 75% w/w.

The pharmaceutical composition can further include an additional excipient, for example, silicified microcrystalline cellulose, microcrystalline cellulose, lactose, compressible sugars, xylitol, sorbitol, mannitol, pregelatinized starch, maltodextrin, calcium phosphate dibasic, calcium phosphate tribasic, calcium carbonate DC, and the like.

Also, in some aspects the pharmaceutical can include a glidant. For example, the glidant can be colloidal silicon dioxide and the like. In some aspects, the colloidal silicon dioxide can be provided in an amount of about 0.1 to about 6.0% w/w. In a preferred embodiment, the colloidal silicon dioxide can be provided in an amount of about 0.1 to about 1.5% w/w.

In some aspects, the composition can include a lubricant, for example, magnesium stearate, calcium stearate, sodium stearyl fumarate, stearic acid, hydrogenated vegetable oil, glyceryl behenate, polyethylene glycol and the like. For example, the lubricant can be sodium stearyl fumarate. In some embodiments, the sodium stearyl fumarate can be provided in an amount of about 0.25 to about 2.0% w/w.

Also, in some aspects, the composition can include a disintegrant or a supplemental binder. The disintegrant can be, in some embodiments, croscarmellose sodium, sodium starch glycolate, crospovidone, microcrystalline cellulose, pregelatinized starch, corn starch, alginic acid, ion exchange resin and the like. For example, in some embodiments, the pharmaceutical composition can contain a “superdisintegrant,” such as, Crospovidone XL, Ac-Di-Sol” (Croscarmellose Sodium) or “Explotab” (Sodium Starch Glycolate), or the like, in an amount of about 1% to about 10% w/w. Additional, in some embodiments, the supplemental binder can be hydroxypropyl cellulose, polyvinylpyrrolidone, methylcellulose, hydroxypropyl methylcellulose, ethylcellulose, sodium carboxy methylcellulose, and the like.

In additional embodiments, the formulations disclosed herein can also comprise artificial or natural sweeteners, flavors, flavor enhancers and colorants. For example, sweeteners and/or flavors can be included to mask the taste of the drug substance. Exemplary sweeteners, include, for example, Aspartame, Sodium Saccharin, Sodium Cyclamate, Aceulfame K Sucralose, and the like. In some embodiments, one or more colorants can be used to compliment the selected flavor (for example, pink for cherry, etc.).

The compositions disclosed herein can be in the form of tablets, chewing gums, lozenges, films, capsules, gel caps, caplets, pellets, beads, or the like. Doxepin can be provided in an amount of about 1 to about 2 mg. In one embodiment, doxepin can be provided in an amount of about 1 mg. In another embodiment, doxepin is provided in an amount of about 3 to about 4 mg. In another aspect, doxepin can be provided in an amount of about 3 mg. In another embodiment, doxepin can be provided in an amount of about 6 to about 7 mg. In one aspect of this embodiment, doxepin can be provided in an amount of about 6 mg. In still another aspect of the embodiment, doxepin can be provided in an amount of about 8 to about 9 mg.

Embodiments of the invention also provide compositions comprising from about 0.5 to about 9 mg doxepin having hardness values of at least 2 Kp. In other embodiments, the compositions have hardness values of at least 4 Kp, or at least 6 Kp or higher. Some embodiments can provide a tablet that can be comprised of from about 0.5 to about 9 mg doxepin that can have a friability value of 1% or less. In other embodiments, the tablet can have a friability value of about 0.75%, of about 0.5% or of about 0.25%.

Embodiments of the invention also provide pharmaceutical compositions that can be comprised of from about 0.5 to about 9 mg doxepin that can have disintegration times of less than 1 minute per USP protocols. In other embodiments, the compositions can have disintegration time of less than 30 seconds, of less than 20 seconds, of less than 10 seconds or of less than 6 seconds.

Another embodiment provides pharmaceutical compositions comprising from about 0.1 to about 9 mg doxepin having at least an 85 percent release of doxepin within 30 minutes using U.S. Pharmacopeia (USP) Apparatus I at 100 rpm (or Apparatus II at 50 rpm) in 0.1 N HCl or Simulated Gastric Fluid USP without enzymes. In other embodiments, the composition has at least an 85 percent release rate at 15 minutes, at least an 85 percent release rate at 10 minutes, at least an 85 percent release rate at 5 minutes, at least a 90 percent release rate at 30 minutes, at least a 95 percent release rate at 30 minutes. In some aspects of this embodiment, the compositions also have at least an 85 percent release of doxepin within 30 minutes using U.S. Pharmacopeia (USP) Apparatus I at 100 rpm (or Apparatus II at 50 rpm) in a pH 4.5 buffer and/or at least an 85 percent release of doxepin within 30 minutes using U.S. Pharmacopeia (USP) Apparatus I at 100 rpm (or Apparatus II at 50 rpm) in a pH 6.8 buffer of Simulated Intestinal Fluid USP without enzymes.

Some embodiments of the invention provide pharmaceutical compositions comprising from about 0.5 to about 9 mg doxepin having at least an 85 percent release of doxepin within 30 minutes using U.S. Pharmacopeia (USP) Apparatus I at 100 rpm (or Apparatus II at 50 rpm) in a pH 4.5 buffer. In other embodiments, the compositions have at least an 85 percent release rate at 15 minutes, at least an 85 percent release rate at 10 minutes, at least an 85 percent release rate at 5 minutes, at least a 90 percent release rate at 30 minutes or at least a 95 percent release rate at 30 minutes.

Another embodiment provides pharmaceutical compositions comprising from about 0.5 to about 9 mg doxepin having at least an 85 percent release of doxepin within 30 minutes using U.S. Pharmacopeia (USP) Apparatus I at 100 rpm (or Apparatus II at 50 rpm) in a pH 6.8 buffer or Simulated Intestinal Fluid USP without enzymes.

Embodiments of the invention also provide pharmaceutical compositions comprising about 0.5 to about 9 mg doxepin having two or more of the following characteristics: a hardness value of at least 2 Kp, a friability value of 1% or less, a disintegration time of less than 1 minute as per USP protocols, at least an 85 percent release of doxepin within 30 minutes using U.S. Pharmacopeia (USP) Apparatus I at 100 rpm (or Apparatus II at 50 rpm) in 0.1 N HCl or Simulated Gastric Fluid USP without enzymes, at least an 85 percent release of doxepin within 30 minutes using U.S. Pharmacopeia (USP) Apparatus I at 100 rpm (or Apparatus II at 50 rpm) in a pH 4.5 buffer, and at least an 85 percent release of doxepin within 30 minutes using U.S. Pharmacopeia (USP) Apparatus I at 100 rpm (or Apparatus II at 50 rpm) in a pH 6.8 buffer or Simulated Intestinal Fluid USP without enzymes.

Another embodiment provides a batch of unit dosage forms, each comprising from about 0.5 to about 9 mg doxepin, and the batch having content uniformity values between about 85% to 115% of label claim. In other embodiments, the batch of unit dosage forms has content uniformity values of between about 90% to 110% of label claim or of between about 95% to 105% of label claim.

In some embodiments the batch of unit dosage forms comprises from about 100,000 to about 10,000,000 units, from about 500,000 to about 5,000,000 units, from about 1,000,000 to about 4,000,000 units, or from about 3,000,000 to about 4,000,000 units. The units can be in the form of tablets, chewing gums, lozenge, films, capsules, caplets, pills, gel caps, pellets, beads, and the likes

Embodiments of the invention also provides a batch of unit dosage forms, each comprising from about 0.5 to about 9 mg doxepin, having a content uniformity percent relative standard deviation of less than 5%. In other embodiments, the batch of unit dosage forms has a content uniformity percent relative standard deviation of less than 4%, less than 3%, less than 2% or less than 1%.

Embodiments of the invention also provide methods of making a plurality of doxepin unit dosage forms. The methods can include, for example, providing an amount of doxepin to obtain a plurality of doxepin tablets, wherein each tablet comprises between about 0.1 mg to 9 mg of doxepin; providing one or more excipients; mixing said doxepin and excipients such that the plurality of doxepin dosage forms comprises at least one of content uniformity values between about 85% and 115% of label claim or a content uniformity percent relative standard deviation of less than 5%. In other embodiments, the plurality of dosage forms comprises content uniformity values between about 90% to 110% of label claim, or between about 95% to 105% of label claim. In other embodiments, the plurality of dosage forms comprises a content uniformity percent relative standard deviation of less than 5%, of less than 4%, of less than 3%, of less than 2%, or of less than 1%. In some embodiments, at least one excipient can be quick dissolve delivery system, such as, for example, a RxCIPIENTS™ FM1000, Pharmaburst, F-Melt™, and the like. Also, in some aspects, a second excipient can be selected from the group consisting of microcrystalline cellulose, lactose, a compressible sugar, xylitol, sorbitol, mannitol, pregelatinized starch, maltodextrin, calcium phosphate dibasic, calcium phosphate tribasic, calcium carbonate DC, a calcium silicate, and the like.

In some embodiments, the invention can provide methods of making a plurality of doxepin unit dosage forms, wherein doxepin is combined with RxCIPIENTS™ FM1000. In a preferred embodiment, the RxCIPIENTS™ FM1000 is provided in an amount of about between about 15% and 30% w/w. The plurality of dosage forms can include from about 100,000 to about 10,000,000 units, from about 500,000 to about 5,000,000 units, from about 1,000,000 to about 4,000,000 units or from about 3,000,000 to about 4,000,000 units.

In some embodiments, the invention can provide methods of making a plurality of doxepin unit dosage forms, wherein doxepin is combined with Pharmaburst. In a preferred embodiment, the Pharmaburst is provided in an amount of between about 50 and about 90% w/w.

In some embodiments, the invention can provide methods of making a plurality of doxepin unit dosage forms, wherein doxepin is combined with F-Melt™. In a preferred embodiment, the F-Melt™ can be provided in an amount of about 25% to about 65% w/w. The plurality of dosage forms can include from about 100,000 to about 10,000,000 units, from about 500,000 to about 5,000,000 units, from about 1,000,000 to about 4,000,000 units or from about 3,000,000 to about 4,000,000 units.

Another embodiment of the invention is directed to a pharmaceutical unit dosage form, that can be comprised of doxepin or a pharmaceutically-acceptable salt thereof in an amount equivalent to about 9 mg doxepin hydrochloride; one or more pharmaceutically-acceptable excipients; and optionally, a capsule or coating. In some embodiments, the excipients and any capsule or coating can be selected to provide a rapid orally disintegrating unit dosage form that is at least externally solid and that has dissolution and bioavailability characteristics such that after administration to a 70 kg human, the dosage form can provide a therapeutically effective plasma concentration of doxepin within a time frame of not more than about 60 minutes. The dosage form can be a tablet, a chewing gum, a lozenge, a film, a capsule, a pill, a caplet, a gel cap, a pellet, a bead, or a dragee. In one embodiment, the dosage form can be an orally disintegrating tablet. In another embodiment, the dosage form can be a capsule. In another embodiment, the time frame to provide a therapeutically effective plasma concentration of doxepin can be less than about 40 minutes. In yet another embodiment, the time frame to provide a therapeutically effective plasma concentration of doxepin can be less than about 20 minutes.

Another embodiment of the invention is directed to a pharmaceutical unit dosage form, that can be comprised of doxepin or a pharmaceutically-acceptable salt thereof in an amount equivalent to about 6 mg doxepin hydrochloride; one or more pharmaceutically-acceptable excipients; and optionally, a capsule or coating. In some embodiments, the excipients and any capsule or coating can be selected to provide a rapid orally disintegrating unit dosage form that can be at least externally solid and that can have dissolution and bioavailability characteristics such that after administration to a 70 kg human, the dosage form can provide a therapeutically effective plasma concentration of doxepin within a time frame of not more than about 60 minutes. The dosage form can be a tablet, a chewing gum, a lozenge, a film, a capsule, a pill, a caplet, a gel cap, a pellet, a bead, or a dragee. In one embodiment, the dosage form can be an orally disintegrating tablet. In another embodiment, the dosage form can be a capsule. In another embodiment, the time frame to provide a therapeutically effective plasma concentration of doxepin can be less than about 40 minutes. In yet another embodiment, the time frame to provide a therapeutically effective plasma concentration of doxepin can be less than about 20 minutes.

Another embodiment of the invention is directed to a pharmaceutical unit dosage form, that can be comprised of doxepin or a pharmaceutically-acceptable salt thereof in an amount equivalent to about 3 mg doxepin hydrochloride; one or more pharmaceutically-acceptable excipients; and optionally, a capsule or coating. In some embodiments, the excipients and any capsule or coating can be selected to provide a rapid orally disintegrating unit dosage form that is at least externally solid and that has dissolution and bioavailability characteristics such that after administration to a 70 kg human, the dosage form can provide a therapeutically effective plasma concentration of doxepin within a time frame of not more than about 60 minutes. The dosage form can be a tablet, a chewing gum, a lozenge, a film, a capsule, a pill, a caplet, a gel cap, a pellet, a bead, or a dragee. In one embodiment, the dosage form is an orally disintegrating tablet. In another embodiment, the dosage form can be a capsule. In another embodiment, the time frame to provide a therapeutically effective plasma concentration of doxepin can be less than about 40 minutes. In yet another embodiment, the time frame to provide a therapeutically effective plasma concentration of doxepin can be less than about 20 minutes.

Another embodiment of the invention is directed to a pharmaceutical unit dosage form, that can comprise of doxepin or a pharmaceutically-acceptable salt thereof in an amount equivalent to about 1 mg doxepin hydrochloride; one or more pharmaceutically-acceptable excipients; and optionally, a capsule or coating. In some embodiments, the excipients and any capsule or coating can be selected to provide a rapid orally disintegrating unit dosage form that is at least externally solid and that has dissolution and bioavailability characteristics such that after administration to a 70 kg human, the dosage form can provide a therapeutically effective plasma concentration of doxepin within a time frame of not more than about 60 minutes. The dosage form can be a tablet, a chewing gum, a lozenge, a film, a capsule, a pill, a caplet, a gel cap, a pellet, a bead, or a dragee. In one embodiment, the dosage form is an orally disintegrating tablet. In another embodiment, the dosage form can be a lozenge. In another embodiment, the dosage form can be a film. In another embodiment, the dosage form can be a chewing gum. In another embodiment, the time frame to provide a therapeutically effective plasma concentration of doxepin can be less than about 40 minutes. In yet another embodiment, the time frame to provide a therapeutically effective plasma concentration of doxepin can be less than about 20 minutes.

Some embodiments encompass, the use of doxepin, or a pharmaceutically acceptable salt, prodrug, or metabolite thereof in the preparation of an orally disintegrable medicament for lessening time to sleep onset in a patient who has insomnia. In some aspects, the orally disintegrable medicament can be formulated to disintegrate in the mouth of a patient in less than 60 seconds without the addition of water. In a preferred aspect, the orally disintegrable medicament can be formulated to disintegrate in the mouth of a patient in less than 30 seconds without the addition of water. In some embodiments, the oral disintegrable medicament can have a friability of less than about 2% when tested per the U.S.P. In a preferred embodiment, oral disintegrable medicament can have a friability of about 0.8% when tested per the U.S.P. Further, in some embodiments, the oral disintegrable medicament can have a hardness of about 15 Newtons or greater. In a preferred embodiment, the oral disintegrable medicament can have a hardness value in the range of about 20 Newtons to about 60 Newtons. In a further preferred embodiment, the oral disintegrable medicament can have a hardness of about 30 Newtons.

Some embodiments encompass, the use of doxepin, or a pharmaceutically acceptable salt, prodrug, or metabolite thereof, wherein the doxepin, or a pharmaceutically acceptable salt, prodrug, or metabolite thereof can comprise a plurality of microparticles of the doxepin, or a pharmaceutically acceptable salt, prodrug, or metabolite thereof and a protective material substantially surrounding the doxepin, or a pharmaceutically acceptable salt, prodrug, or metabolite thereof. For example, in some aspects, the microparticles can consist essentially of particles from about 75 microns to about 600 microns. Further, in some aspects, the microparticles can comprise about 0.01 to about 75% by weight of the orally disintegrable medicament. Additionally, the oral disintegrable medicament can further comprise a matrix with a non-direct compression filler and a lubricant. For example, the non-direct compression filler can be a non-direct compression sugar or non-direct compression sugar alcohol. In some embodiments, the non-direct compression filler can have an average particle size of about 90 microns or less. Also, in some embodiments, the oral disintegrable medicament can further comprise an effervescent agent.

In some embodiments, the orally disintegrating form of doxepin, or a pharmaceutically acceptable salt, prodrug, or metabolite thereof, can be for use in lessening sleep onset in a patient being treated for insomnia, wherein the orally disintegrating form can be formulated to disintegrate in the mouth of a patient in less than about 60 seconds without water intake; and wherein the orally disintegrating form can be associated with achieving a plasma concentration of doxepin faster than an oral dosage that is not oral disintegrating. In some embodiments, a therapeutically effective plasma concentration of doxepin can be achieved in less than 60 minutes. In some embodiments, a therapeutically effective plasma concentration of doxepin can be achieved in less than 40 minutes. In some embodiments, a therapeutically effective plasma concentration of doxepin can be achieved in less than 20 minutes. In some aspects, the orally disintegrating form can be formulated to disintegrate in the mouth of the patient in a range of about 5 seconds to about 30 seconds. In some embodiments, the orally disintegrating form can have a friability of less than about 2% when U.S.P. tested. In a preferred embodiment, the orally disintegrating form has a friability of about 0.8% when U.S.P. tested. Further, in some embodiments, the orally disintegrating form can have a hardness of about 20 Newtons or greater. In a preferred embodiment, the orally disintegrating form can have a hardness in the range of about 20 Newtons to about 60 Newtons. In a further preferred embodiment, orally disintegrating form can have a hardness of about 30 Newtons.

In some embodiments, the orally disintegrating form further can include an effervescent agent. In some aspects, the effervescent agent generates evolved gas that can have a volume of about 5 cm³ to about 30 cm³. In some embodiments, the effervescent agent can include, but is not limited to, an acid source and a carbonate source, thereby generating a gas. The gas can be carbon dioxide or oxygen.

Another embodiment provides a method of treating insomnia, comprising identifying an individual in need of such treatment, and administering any of the compositions disclosed herein to the individual.

Yet another embodiment provides a method of enhancing sleep maintenance, comprising identifying an individual in need of such enhancement, and administering any of the compositions disclosed herein to the individual.

Some embodiments relate to methods of treating insomnia, which methods can include providing a patient treated with a conventional formulation of a sleep aid and treating said patient with a fast disintegrating form of doxepin, or a pharmaceutically acceptable salt, prodrug, or metabolite thereof such that time to sleep onset can be reduced.

Some embodiments relate to methods of treating insomnia, which methods can include providing a patient treated with a conventional formulation of a sleep aid treating said patient with a fast disintegrating formulation form of doxepin, or a pharmaceutically acceptable salt, prodrug, or metabolite thereof under conditions such that sleep duration can be maintained into the 8^{th} hour.

Some embodiments relate to methods of treating insomnia, which methods can include providing a patient treated with a conventional formulation of a sleep aid treating said patient with a fast disintegrating formulation form of doxepin, or a pharmaceutically acceptable salt, prodrug, or metabolite thereof under conditions awakenings after sleep onset are decreased.

Some embodiments relate to methods of treating insomnia that can be comprised of treating a patient with a fast disintegrating formulation form of doxepin, or a pharmaceutically acceptable salt, prodrug, or metabolite thereof under conditions such that sleep duration can be maintained into the 8^{th} hour.

Some embodiments relate to methods of treating insomnia that can be comprised of treating a patient with a fast disintegrating formulation form of doxepin, or a pharmaceutically acceptable salt, prodrug, or metabolite thereof under conditions such that awakenings after sleep onset can be decreased.

### BRIEF DESCRIPTION OF THE DRAWINGS

FIG. 1 is graph simulating absorption of doxepin in the blood plasma over two hours.

### DETAILED DESCRIPTION OF THE INVENTION

Embodiments of the invention generally relate to new and surprisingly effective doxepin formulations and methods of using low-dose forms of doxepin, including, for example, use in the treatment of insomnia. For example, some preferred embodiments relate to buccal, sublingual and fastmelt doxepin formulations, as well as methods of using the same. Also, some embodiments of this invention relate to novel and economic methods of manufacturing low-dose dosage forms of doxepin, pharmaceutically acceptable salts thereof, or other doxepin-related compounds.

It has only recently been appreciated that the use of doxepin to treat sleep disorders, in particular low dose doxepin, can be affected by the ability of administered drug to enter the blood stream. When doxepin is taken orally (e.g., tablets and capsules) and swallowed much of the drug is transported directly to the liver before it can enter the bloodstream. As a result, some of the drug is metabolized in the liver before reaching the bloodstream or the intended site in the body. Furthermore, swallowed doxepin also may be degraded in the gastrointestinal tract. Accordingly, for the first time it has been recognized herein that it can be preferable to formulate doxepin for sleep in order to avoid the first pass liver effect and the degradation effects in the stomach. Thus, some embodiments relate to doxepin formulations that can be administered orally, sublingually, buccally so that more of the drug can be taken into the bloodstream before it is metabolized by the liver or degraded in the stomach.

Doxepin is a tricyclic compound currently approved for treatment of depression or anxiety at a daily dose of 75 mg to 300 mg. Doxepin is marketed under the commercial name SINEQUAN® and in generic form, and can be obtained in the United States generally from pharmacies in capsule form in amounts of 10, 25, 50, 75, 100 and 150 mg dosage, and in liquid concentrate form at 10 mg/mL. The capsule formulations contain Doxepin HCl with cornstarch and magnesium stearate/sodium lauryl sulfate. Capsule shells can also contain gelatin, sodium lauryl sulfate, sodium metabisulfate and colorants.

The use of low dose doxepin for the treatment of insomnia is described in U.S. Pat. Nos. 5,502,047 and 6,211,229, the entire contents of which are incorporated herein by reference. As mentioned above, many individuals currently suffer from sleep disorders, such as insomnia. There is a need for improved compositions and methods for treating such individuals.

### Compounds

### Doxepin:

Doxepin HCl is a tricyclic compound currently approved and available for treatment of depression and anxiety. Doxepin has the following structure:

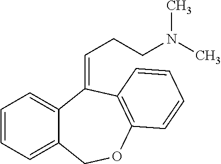 [SMILES: CN(C)CC/C=C1/c2ccccc2COc2ccccc21]

For all compounds disclosed herein, unless otherwise indicated, where a carbon-carbon double bond is depicted, both the cis and trans stereoisomers, as well as mixtures thereof are encompassed.

Doxepin can be depicted as follows:

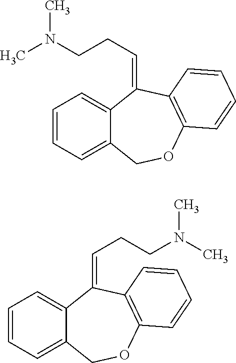 [SMILES: CN(C)CC/C=C1\c2ccccc2COc2ccccc21 ; CN(C)CC/C=C1/c2ccccc2COc2ccccc21]

Doxepin belongs to a class of psychotherapeutic agents known as dibenzoxepin tricyclic compounds, and is currently approved and prescribed for use as an antidepressant to treat depression and anxiety. Doxepin has a well-established safety profile, having been prescribed for over 35 years.

It is contemplated that doxepin for use in the compositions and methods described herein can be obtained from any suitable source or made by any suitable method. For example, doxepin HCl can be obtained from Plantex Ltd. (DMF No. 3230). In the Biopharmaceutic Classification System, doxepin HCl, USP is designated as a Class One compound, with high solubility and high permeability (Wu-Benet, 2005). The Plantex-supplied doxepin HCl, USP has a particle size specification of not less than 80% smaller than 38 microns and not less than 90% smaller than 125 microns as measured by an Air Jet Sieve method. Also, doxepin can be prepared according to the method described in U.S. Pat. Nos. 3,438,981, 3,420,851, and 3,420,851 all of which are incorporated herein by reference in their entirety. It should be noted and understood that although many of the embodiments described herein specifically refer to “doxepin,” other doxepin-related compounds can also be used, including, for example, pharmaceutically acceptable salts, prodrugs, metabolites, in-situ salts of doxepin formed after administration, and solid state forms, including polymorphs and hydrates.

### Metabolites:

In addition, doxepin metabolites can be prepared and used. By way of illustration, some examples of metabolites of doxepin can include, but are not limited to, doxepin, hydroxydoxepin, hydroxyl-N-doxepin, doxepin N-oxide, N-acetyl-N-doxepin, N-desmethyl-N-formyldoxepin, quaternary ammonium-linked glucuronide, 2-O-glucuronyldoxepin, didoxepin, 3-O-glucuronyldoxepin, or N-acetyldidoxepin. The metabolites of doxepin can be obtained or made by any suitable method, including the methods described above for doxepin.

Doxepin has the following structure:

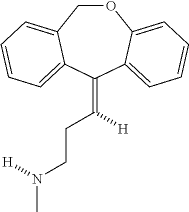

Doxepin is commercially available as a forensic standard. For example, it can be obtained from Cambridge Isotope Laboratories, Inc. (50 Frontage Road, Andover, Mass.). Doxepin for use in the methods discussed herein can be prepared by any suitable procedure. For example, doxepin can be prepared from 3-methylaminopropyl triphenylphosphonium bromide hydrobromide and 6,11-dihydrodibenz(b,e)oxepin-11-one according to the method taught in U.S. Pat. No. 3,509,175, which is incorporated herein by reference in its entirety. As another example, doxepin can be from doxepin hydrochloride as taught in U.S. Pat. No. 5,332,661, which is incorporated herein by reference in its entirety.

Hydroxydoxepin has the following structure:

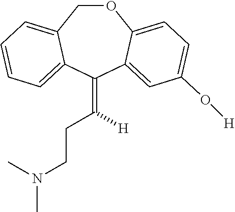

2-Hydroxydoxepin can be prepared by any suitable method, including as taught by Shu et al. (Drug Metabolism and Disposition (1990) 18:735-741), which is incorporated herein by reference in its entirety.

Hydroxyl-N-doxepin has the following structure:

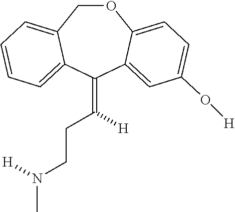

2-Hydroxy-N-doxepin can be prepared any suitable method.

Doxepin N-oxide has the following structure:

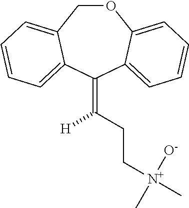

Doxepin-N-oxide can be prepared by any suitable method. For example, doxepin-N-oxide can be prepared as taught by Hobbs (Biochem Pharmacol (1969) 18:1941-1954), which is hereby incorporated by reference in its entirety.

N-acetyl-N-doxepin has the following structure:

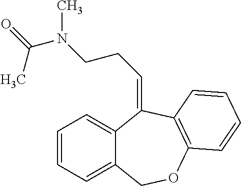 [SMILES: CC(=O)N(C)CC/C=C1\c2ccccc2COc2ccccc21]

N-acetyl-N-doxepin can be prepared by any suitable means. For example, N-acetyl-N-doxepin has been produced in filamentous fungus incubated with doxepin as taught by Moody et al. (Drug Metabolism and Disposition (1999) 27:1157-1164), hereby incorporated by reference in its entirety.

N-desmethyl-N-formyldoxepin has the following structure:

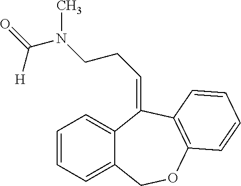 [SMILES: CN(C=O)CC/C=C1\c2ccccc2COc2ccccc21]

N-desmethyl-N-formyldoxepin can be prepared by any suitable means. For example, N-desmethyl-N-formyldoxepin has been produced in filamentous fungus incubated with doxepin as taught by Moody et al. (Drug Metabolism and Disposition (1999) 27:1157-1164), hereby incorporated by reference in its entirety.

N-acetyldidoxepin has the following structure:

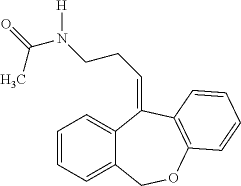 [SMILES: CC(=O)NCC/C=C1\c2ccccc2COc2ccccc21]

N-acetyldidoxepin can be prepared by any suitable means. For example, N-acetyldidoxepin has been produced in filamentous fungus incubated with doxepin as taught by Moody et al. (Drug Metabolism and Disposition (1999) 27:1157-1164), hereby incorporated by reference in its entirety.

Didoxepin has the following structure:

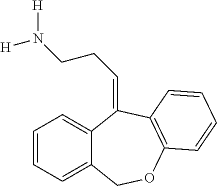 [SMILES: NCC/C=C1\c2ccccc2COc2ccccc21]

Didoxepin can be prepared by any suitable means. For example, didoxepin has been isolated from plasma and cerebrospinal fluid of depressed patients taking doxepin, as taught by Deuschle et al. (Psychopharmacology (1997) 131:19-22), hereby incorporated by reference in its entirety.

3-O-glucuronyldoxepin has the following structure:

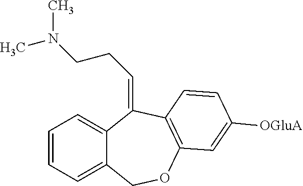

3-O-glucuronyldoxepin can be prepared by any suitable means. For example, 3-O-glucuronyldoxepin has been isolated from the bile of rats given doxepin, as described by Shu et al. (Drug Metabolism and Disposition (1990)18:1096-1099), hereby incorporated by reference in its entirety.

2-O-glucuronyldoxepin has the following structure:

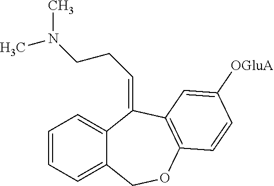

2-O-glucuronyldoxepin can be prepared by any suitable means. For example, 2-O-glucuronyldoxepin has been isolated from the bile of rats given doxepin, and also in the urine of humans given doxepin, as described by Shu et al. (Drug Metabolism and Disposition (1990) 18:1096-1099), hereby incorporated by reference in its entirety.

Quaternary ammonium-linked glucuronide of doxepin (doxepin N⁺-glucuronide) has the following structure:

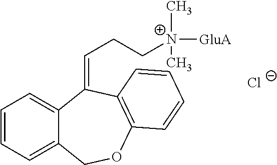

N⁺-glucuronide can be obtained by any suitable means. For example, doxepin N⁺-glucuronide can be prepared, and can be isolated as taught by Luo et al. (Drug Metabolism and Disposition, (1991) 19:722-724), hereby incorporated by reference in its entirety.

### Pharmaceutically Acceptable Salts:

As mentioned above, the methods and other embodiments described herein can utilize any suitable pharmaceutically acceptable salt or prodrug of doxepin or salts or prodrugs of doxepin metabolites. Therefore, the substitution or use in combination of salts and prodrugs is specifically contemplated in the embodiments described herein. The pharmaceutically acceptable salts and prodrugs can be made by any suitable method.

The term “pharmaceutically acceptable salt” refers to an ionic form of a compound that does not cause significant irritation to an organism to which it is administered and does not abrogate the biological activity and properties of the compound. Pharmaceutical salts can be obtained by reacting a compound of the invention with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glutamine, tris(hydroxymethyl)methylamine, and salts with amino acids such as arginine, lysine, and the like. Pharmaceutically acceptable salts are more fully described in the following paragraph.

The acids that can be used to prepare pharmaceutically acceptable acid addition salts include, for example, those that form non-toxic acid addition salts, i.e., salts containing pharmacologically acceptable anions, such as the acetate, benzenesulfonate, benzoate, bicarbonate, bisulfate, bitartrate, borate, bromide, calcium edetate, camsylate, carbonate, chloride, clavulanate, citrate, dihydrochloride, edetate, dislyate, estolate, esylate, ethylsuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsulfate, mucate, napsylate, nitrate, oleate, oxalate, pamoate (embonate), palmitate, pantothenate, phospate/diphosphate, polygalacturonate, salicylate, stearate, subacetate, succinate, tannate, tartrate, teoclate, tosylate, triethiodode, and valerate salts.

The bases that can be used to prepare pharmaceutically acceptable base addition salts include, for example, those that form non-toxic base addition salts, i.e., base salts formed with metals or amines, such as alkali and alkaline earth metals or organic amines. Non-limiting examples of metals used as cations include sodium, potassium, magnesium, calcium, and the like. Also included are heavy metal salts such as for example silver, zinc, cobalt, and cerium. Non-limiting examples of suitable amines include N,N′-dibenzylethylenediamine, chloroprocaine, choline, diethanolamine, ethylenediamene, N-methylglucamine, and procaine.

### Prodrugs:

The term “prodrug” refers to an agent that is converted into the active drug in vivo. Prodrugs are often useful because, in some situations, they can be easier to administer than the active drug. They can, for instance, be bioavailable by oral administration whereas the active drug is not. The prodrug may also have improved solubility in pharmaceutical compositions over the active drug. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. Examples of prodrug groups can be found in, for example, T. Higuchi and V. Stella, in “Pro-drugs as Novel Delivery Systems,” Vol. 14, A.C.S. Symposium Series, American Chemical Society (1975); H. Bundgaard, “Design of Prodrugs,” Elsevier Science, 1985; and “Bioreversible Carriers in Drug Design: Theory and Application,” edited by E. B. Roche, Pergamon Press: New York, 14-21 (1987), each of which is hereby incorporated by reference in its entirety.

### Compositions

Dosage form development can require the selection of excipients based on the properties of the drug substance being formulated. Several preferred embodiments of this invention are provided. These should not be construed as limiting the scope of this invention.

Some embodiments of the invention are based upon the new discovery of physical characteristics and challenges associated with low-dose doxepin compositions, such as, uniformity and/or potency, and also upon a new understanding of pharmacokinetics of doxepin when it is used to treat sleep disorders. For example, in a preferred embodiment, the target pharmacokinetic profile for the orally disintegrating formulation is designed to preserve the pharmacokinetic profile of a standard oral dosage form (i.e., maintenance and duration) but with a more rapid onset.

For example, it has been found that formulation of compositions at the lower dose range can present a considerable challenge in maintaining consistent potency and uniformity in the drug product manufacturing process, while also maintaining a high yield. For example, assuring the homogeneity of the powder blend for production of low-dose dosage forms can represent a major quality assurance consideration. The selection of the particular excipient or excipients used, and how to properly blend and prevent non-uniformity and segregation were based upon previously unrecognized characteristics and needs for doxepin formulation, particularly low-dose formulations.

Also, in some embodiments, the compositions are based upon previously unknown pharmacokinetics of low-dose doxepin for sleep. Although doxepin dissolves quickly in the stomach, it undergoes a significant first pass extraction or metabolism in the liver. Thus, it can take some time for the sleep promoting action of the drug to take place. No one previously recognized the sleep pharmacokinetics of doxepin, such as, sleep onset characteristics of doxepin; and for sleep, even decreasing induction time by a few minutes can provide an enormous benefit. In the context of sleep, early onset of drug action can be important due to the discreet window of time (e.g., 8 hours) in which a patient needs to sleep. As a consequence, some embodiments relate to compositions that can contribute to accelerated action of the drug. That need was not recognized previously, in particular for depression and anxiety where there was no need for fast onset due to the ongoing and chronic nature of those conditions. The unique needs of doxepin for treating sleep were not appreciated in the prior art.

Accordingly, some embodiments relate to compositions for the treatment of such disorders where careful selection of excipients was used to address the previously unrecognized characteristics of low-dose doxepin and doxepin for use in treating sleep disorders. Described below and elsewhere herein are new and unexpectedly effective doxepin formulations.

Doxepin HCl, USP, is a white crystalline powder with a slight amine-like odor supplied by Plantex Ltd. In the Biopharmaceutic Classification System, doxepin HCl, USP is designated as a Class One compound, with high solubility and high permeability (Wu-Benet, 2005). The Plantex-supplied doxepin HCl, USP has a particle size specification of not less than 80% smaller than 38 microns and not less than 90% smaller than 125 microns as measured by an Air Jet Sieve method.

In a preferred embodiment, the compositions disclosed herein can include from about 0.01 mg to about 9 mg of doxepin, or from about 0.5 mg to about 7 mg doxepin, or from about 1 mg to about 6 mg doxepin. In some embodiments, the compositions include from about 0.5 mg to about 2 mg doxepin, or from about 2.5 mg to about 4 mg, or from about 5.9 mg to about 7 mg doxepin.

As discussed above, in some embodiments, low-dose metabolites of doxepin, doxepin prodrugs or pharmaceutically acceptable salts of doxepin, or other doxepin-related compounds, can be used in place of, or in addition to, low-dose doxepin in the formulations described herein.

Some embodiments provide low-dose doxepin tablets, lozenges, chewing gum, capsules, caplets, pills, gel caps, pellets, beads, or dragee dosage forms that can be absorbed across the oral mucosa. Some embodiments specifically exclude one or more such dosage forms.

Preferably, the formulations disclosed herein can provide favorable drug processing qualities, including, for example, but not limited to, rapid tablet press speeds, reduced compression force, reduced ejection forces, blend uniformity, content uniformity, uniform dispersal of color, accelerated disintegration time, rapid dissolution, low friability (preferable for downstream processing such as packaging, shipping, pick-and-pack, etc.) and dosage form physical characteristics (e.g., weight, hardness, thickness, friability) with little variation. Many of these qualities, notably, content uniformity and blend uniformity, are difficult to obtain in low dose formulations.

In one embodiment the invention described herein provides a hard, compressed, fast disintegrating tablet adapted for oral administration, preferably for oral transmucosal administration. The tablet can include particles made of an active ingredient and a protective material. These particles can be provided in an amount of between about 0.01 and about 75% by weight based on the weight of the tablet, for example. The tablet can also include a matrix made from a nondirect compression filler, a wicking agent, and a hydrophobic lubricant. The preferred tablet matrix can comprise, for example, at least about 60% rapidly water-soluble ingredients based on the total weight of the matrix material. The preferred tablet can have, for example, a hardness of between about 2 Kp to about 6 Kp, a friability of less than 0.8% when measured by U.S.P. and is adapted to disintegrate spontaneously in the mouth of a patient in less than about 60 seconds (and, more preferably, less than about 30 seconds) thereby liberating said particles. Preferably, the tablets are capable of being stored in bulk.

In another embodiment the invention described herein provides a compressed fast disintegrating tablet comprising effervescent agents. Examples of effervescent pharmaceutical compositions suitable for use and adaptation in conjunction with some of the present embodiments include the compositions described in Pather, U.S. Pat. No. 6,200,604, which is incorporated herein by reference in its entirety.

Other pharmaceutical compositions suitable for use and adaptation in conjunction with the compositions disclosed herein are described in U.S. Pat. No. 5,178,878 to Wehling, et al., U.S. Pat. No. 5,223,264 to Wehling, et al. and U.S. Pat. No. 6,024,981 to Khankari et al., each of which is hereby incorporated by reference in its entirety.

In one embodiment, the invention described herein provides a dosage form as a fast disintegrating ordered-mixture composition as disclosed in European patent EP 0 324 725 (herein incorporated by reference). In this embodiment, doxepin covers (in a finely dispersed state) the surface of substantially larger carrier particles. Such compositions disintegrate rapidly in water, thereby dispersing their contents of microscopic drug particles.

In one embodiment the present invention describes a hard, compressed, fast disintegrating tablet, formulated with an antidepressant, adapted for oral administration. The tablet can include particles made of an active ingredient and a protective material. These particles can be provided in an amount of between about 0.01 and about 75% by weight based on the weight of the tablet. The tablet can also include a matrix made from a nondirect compression filler, a wicking agent, and a lubricant. The preferred tablet matrix comprises at least about 60% rapidly water-soluble ingredients based on the total weight of the matrix material. The preferred tablet has a hardness of between about 20 and about 60 Newtons, a friability of less than 1% when measured by U.S.P. and is adapted to disintegrating spontaneously in the mouth of a patient in less than about 60 seconds (and, more preferably, less than about 30 seconds) and thereby liberate said particles and be capable of being stored in bulk.

In one embodiment, the invention described herein provides a method for reducing time to sleep onset comprising providing a patient treated with a fast disintegrating formulation of doxepin under conditions such that time to sleep onset is reduced. In a preferred embodiment, said fast disintegrating doxepin formulation is an ODT.

In another embodiment, the invention provides a method for maintaining sleep for 8 hours comprising providing a patient treated with a fast disintegrating formulation of doxepin under conditions such that sleep time duration is maintained for 7 to 8 hours. In a preferred embodiment, said fast disintegrating doxepin formulation is an orally disintegrating tablet (ODT).

In still another embodiment, the invention described herein provides a method for reducing awakening after sleep onset comprising providing a patient treated with a fast disintegrating formulation of doxepin under conditions such that awakenings are reduced after sleep onset. In a preferred embodiment said fast disintegrating doxepin formulation is an ODT.

In preferred embodiments, a quick dissolve delivery system is used in the orally disintegrating formulations described herein. Preferably, the quick dissolve delivery system is a directly compressible excipient for orally disintegrating tablet formulations. For example, the quick dissolve excipient can be, but is not limited to, RxCIPIENTST™ FM1000, Pharmaburst, or F-Melt™. In some embodiments, the orally disintegrating tablet can dissolve within 5 minute. In a preferred embodiment, the orally disintegrating tablet can dissolve within 1 minute. In some embodiments, the amount of doxepin in the formulation can be from about 0.1 to about 9 mg.

In some embodiments, the compositions disclosed herein can include doxepin in the amount of about 0.1 to 20% of the total formulation amount and Pharmaburst in the amount of about 50% to about 90%, or from about 60% to about 85%, or from about 70% to about 80% of the total formulation amount. In some embodiments, the amount of doxepin in the formulation can be from about 0.1 to about 9 mg. In some embodiments, the amount of doxepin can be 3 mg (3%) and the amount of Pharmaburst can be 85 mg (85%) in a 100 mg tablet. In some embodiments, the amount of doxepin can be 3 mg (4%) and the amount of Pharmaburst can be 61.8 mg (82.4%) in a 75 mg tablet. In a preferred embodiment, the amount of doxepin can be 1 mg (1%) and the amount of Pharmaburst can be 87 mg (87%) in a 100 mg tablet. In a further preferred embodiment, the amount of doxepin can be 1 mg (1.3%) and the amount of Pharmaburst can be 61.8 mg (82.4%) in a 75 mg tablet.

In some embodiments, the compositions disclosed herein can include RxCIPIENTS™ FM1000 in the amount of about 15 to about 35%, or from about 20% to about 30% of the total formulation amount and doxepin in the amount of about 0.1 to 20% of the total formulation. In some embodiments, the amount of doxepin in the formulation can be from about 0.1 to about 9 mg. In some embodiments, the amount of doxepin in the can be 3 mg (3%) and the amount of RxCIPIENTS™ FM1000 can be 29.4 mg (29.4%) in a 100 mg tablet. In some embodiments, the amount of doxepin can be 3 mg (4%) and the amount of RxCIPIENTS™ FM1000 can be 22.05 mg (29.4%) in a 75 mg tablet. In some embodiments, the amount of doxepin can be 1 mg (1%) and the amount of RxCIPIENTS™ FM1000 can be 32.4 mg (32.4%) in a 100 mg tablet. In some embodiments, the amount of doxepin can be 1 mg (1.3%) and the amount of RxCIPIENTS™ FM1000 can be 22.05 mg (29.4%) in a 75 mg tablet.

In some embodiments, the compositions disclosed herein can comprise F-Melt™ as the excipient in the amount of about 25 to 65%, or from about 30% to about 62%, or from about 40% to about 60% of the total formulation amount and doxepin in the amount of about 0.1 to 20% of the total formulation amount. In some embodiments, the amount of doxepin in the formulation can be from about 0.1 to about 9 mg. In some embodiments, the amount of doxepin in the can be 3 mg (3%) and the amount of F-Melt™ can be 58 mg (58%) in a 100 mg tablet. In some embodiments, the amount of doxepin can be 3 mg (4%) and the amount of F-Melt™ can be 43.4 mg (58%) in a 75 mg tablet. In some embodiments, the amount of doxepin can be 1 mg (1%) and the amount of F-Melt™ can be 60 mg (60%) in a 100 mg tablet. In some embodiments, the amount of doxepin can be 1 mg (1.3%) and the amount of F-Melt™ can be 415 mg (58%) in a 75 mg tablet.

In some embodiments, the doxepin compositions disclosed herein can be formulated as disclosed in Drug Delivery to the Oral Cavity: Molecules to Market, Ghosh and Pfister, eds., 2005, Taylor & Francis, which is hereby incorporated by reference in its entirety.

In some embodiments, doxepin can be formulated using any commercially available orally disintegrating technologies such as, for example, but not limited to, FlashDose®, Zydis, OraSolv, DuraSolv, FlastTab, AdvaTab™, OraQuick®, Lyoc®, SATAB, WOWTAB, and the like.

Making the drug available for absorption with minimal delay can be important in the treatment of medical conditions such as insomnia. In a preferred embodiment, the formulations can yield extremely rapid disintegration times of 1 minute or less as per USP protocols. Preferably, the formulation yields disintegration times of 60, 50, 40, 30, 25 seconds or less.

In other embodiments, the formulation yields a rapidly orally disintegrating dosage form, for which greater than 85% of the doxepin containing dosage form dissolves within 5 minutes. In some embodiments the formulation yields a rapidly orally dissolving dosage form, for which 65% to 85% of the doxepin containing dosage form dissolves within 5 minutes. In some embodiments, the formulation yields a rapidly orally dissolving dosage form, for which 45% to 65% of the doxepin containing dosage form dissolves within 5 minutes. In some embodiments, the formulation yields a rapidly orally dissolving dosage form, for which 25% to 45% of the doxepin containing dosage form dissolves within 5 minutes. In some embodiments, the formulation yields a rapidly orally disintegrating dosage form, for which 5% to 25% of the doxepin containing dosage form dissolves within 5 minutes.

In some embodiments, the formulation yields a rapidly orally disintegrating dosage form, for which 5% to 25% of the doxepin containing dosage form dissolves within 1 minute. In a preferred embodiment, the formulation yields a rapidly orally dissolving dosage form, for which 25% to 45% of the doxepin containing dosage form dissolves within 1 minute. In another preferred embodiment, the formulation yields a rapidly orally dissolving dosage form, for which 45% to 85% of the doxepin containing dosage form dissolves within 1 minute. In further preferred embodiments, the formulation yields a rapidly orally dissolving dosage form, for which 85% to 99% of the doxepin containing dosage form dissolves within 1 minute. In some aspects nearly 100% of the rapidly orally disintegrating dosage form containing doxepin dissolves within 1 minute.

In other embodiments, the formulation yields a rapidly orally disintegrating dosage form, for which greater than 85% of the doxepin is absorbed across the oral mucosa within 5 minutes. In some embodiments the formulation yields a rapidly orally disintegrating dosage form, for which 65% to 85% of the doxepin is absorbed across the oral mucosa within 5 minutes. In some embodiments, the formulation yields a rapidly orally disintegrating dosage form, for which 45% to 65% of the doxepin is absorbed across the oral mucosa within 5 minutes. In some embodiments, the formulation yields a rapidly orally disintegrating dosage form, for which 25% to 45% of the doxepin is absorbed across the oral mucosa within 5 minutes. In some embodiments, the formulation yields a rapidly orally disintegrating dosage form, for which 5% to 25% of the doxepin is absorbed across the oral mucosa within 5 minutes.

In some embodiments, the formulation yields a rapidly orally disintegrating dosage form, for which 5% to 25% of the doxepin is absorbed across the oral mucosa within 1 minute. In a preferred embodiment, the formulation yields a rapidly orally disintegrating dosage form, for which 25% to 45% of the doxepin is absorbed across the oral mucosa within 1 minute. In another preferred embodiment, the formulation yields a rapidly orally disintegrating dosage form, for which 45% to 85% of the doxepin is absorbed across the oral mucosa within 1 minute. In further preferred embodiments, the formulation yields a rapidly orally disintegrating dosage form, for which 85% to 99% of the doxepin is absorbed across the oral mucosa within 1 minute. In some aspects nearly 100% of the doxepin is absorbed across the oral mucosa within 1 minute.

In other embodiments, a therapeutic blood plasma level of doxepin is achieved within 40 minutes of administering a rapidly orally disintegrating dosage containing 9 mg of doxepin. In other embodiments, a therapeutic blood plasma level of doxepin is achieved within 20 minutes of administering a rapidly orally disintegrating dosage containing 9 mg of doxepin. In other embodiments, a therapeutic blood plasma level of doxepin is achieved within 40 minutes of administering a rapidly orally disintegrating dosage containing 6 mg of doxepin. In other embodiments, a therapeutic blood plasma level of doxepin is achieved within 20 minutes of administering a rapidly orally disintegrating dosage containing 6 mg of doxepin. In other embodiments, a therapeutic blood plasma level of doxepin is achieved within 40 minutes of administering a rapidly orally disintegrating dosage containing 3 mg of doxepin. In other embodiments, a therapeutic blood plasma level of doxepin is achieved within 20 minutes of administering a rapidly orally disintegrating dosage containing 3 mg of doxepin. In other embodiments, a therapeutic blood plasma level of doxepin is achieved within 40 minutes of administering a rapidly orally disintegrating dosage containing 1 mg of doxepin. In other embodiments, a therapeutic blood plasma level of doxepin is achieved within 20 minutes of administering a rapidly orally disintegrating dosage containing 1 mg of doxepin. In other embodiments, a therapeutic blood plasma level of doxepin is achieved within 40 minutes of administering a rapidly orally disintegrating dosage containing 0.5 mg of doxepin. In other embodiments, a therapeutic blood plasma level of doxepin is achieved within 20 minutes of administering a rapidly orally disintegrating dosage containing 0.5 mg of doxepin.

In a preferred embodiment, the low-dose dosage forms described herein are formulated to yield two or more favorable drug characteristics.

The compounds can be formulated readily, for example, by combining the drug substance with any suitable pharmaceutically acceptable excipient for example, but not limited to, binders, diluents, disintegrants, lubricants, fillers, carriers, and the like, as set forth below. Such compositions can be prepared for storage and for subsequent processing.

Acceptable excipients for therapeutic use are well known in the pharmaceutical art, and are described, for example, in Handbook of Pharmaceutical Excipients, 5th edition (Raymond C Rowe, Paul J Sheskey and Sian C Owen, eds. 2005), and Remington: The Science and Practice of Pharmacy, 21st edition (Lippincott Williams & Wilkins, 2005), each of which is hereby incorporated in its entirety. The term “carrier” material or “excipient” herein can mean any substance, not itself a therapeutic agent, used as a carrier and/or diluent and/or adjuvant, or vehicle for delivery of a therapeutic agent to a subject or added to a pharmaceutical composition to improve its handling or storage properties or to permit or facilitate formation of a dose unit of the composition into a discrete article such as a tablet, lozenge, chewing gum, capsule, caplet, gel cap, pill, pellet, bead, and the like suitable for oral administration. Excipients can include, by way of illustration and not limitation, diluents, disintegrants, binding agents, adhesives, wetting agents, polymers, lubricants, glidants, substances added to mask or counteract a disagreeable taste or odor, flavors, colorants, fragrances, and substances added to improve appearance of the composition.

Acceptable excipients include, for example, but are not limited to, RxCIPIENTS™ FM1000, Pharmaburst, F-Melt™, Starch 1500®, microcrystalline cellulose, lactose, sucrose, starch powder, maize starch or derivatives thereof, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinyl-pyrrolidone, and/or polyvinyl alcohol, saline, dextrose, mannitol, lactose, lecithin, albumin, sodium glutamate, cysteine hydrochloride, and the like. Examples of suitable excipients for soft gelatin capsules include vegetable oils, waxes, fats, semisolid and liquid polyols. Suitable excipients for the preparation of solutions and syrups include, without limitation, water, polyols, sucrose, invert sugar and glucose. The compound can also be made in microencapsulated form. If desired, absorption enhancing preparations (for example, liposomes), can be utilized.

It is preferred to use quick dissolve delivery systems, or specialty excipients for orally disintegrating formulations, such as, for example, but not limited to, RxCIPIENTS™ FM1000, Pharmaburst, F-Melt™, and the like. Other such systems or orally disintegrating excipients will be apparent to the skilled artisan.

The compositions and formulations can include any other agents that provide improved transfer, delivery, tolerance, and the like. These compositions and formulations can include, for example, powders, pastes, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax.

Any of the foregoing mixtures can be appropriate in treatments and therapies in accordance with the invention disclosed herein, provided that the active ingredient in the formulation is not inactivated by the formulation; and the formulation is physiologically compatible and tolerable with the route of administration. See also Baldrick P. “Pharmaceutical excipient development: the need for preclinical guidance.” Regul. Toxicol. Pharmacol. 32(2):210-8 (2000), Charman W N “Lipids, lipophilic drugs, and oral drug delivery-some emerging concepts.” J Pharm Sci 0.89(8):967-78 (2000), and the citations therein for additional information related to formulations, excipients and carriers well known to pharmaceutical chemists.

In some embodiments, one or more, or any combination of the listed excipients can be specifically included or excluded from the formulations and/or methods disclosed herein. For example, in some embodiments, microcrystalline cellulose can be specifically excluded.

Oral administration can be accomplished using orally administered formulations, for example, tablets, chewing gums, lozenges, films, capsules, gel caps, caplets, pellets, beads, pills, and the like. In addition, stabilizers can be added. All formulations for oral administration should be in dosages suitable for such administration.

As will be appreciated by those of skill in the art, the amounts of excipients will be determined by drug dosage and dosage form size. In some embodiments disclosed herein, the dosage form size is 100 mg. This dosage form weight is arbitrary and one skilled in the art will realize that a range of weights can be made and are encompassed by this invention. The preferred dosage form range is 50 mg to 200 mg, more preferably 50 mg to 150 mg, more preferably 75 to 100 mg, with the preferred dosage form weight being 100 mg.

In a preferred embodiment, low doses of doxepin are combined with any commercially available quick dissolve delivery system, such as, for example, but not limited to, RxCIPIENTS™ FM1000, Pharmaburst, or F-Melt™, and the like. For example, based on a 100 mg dosage form weight, the range of drug substance is from about 0.75% to about 4.5% w/w and the range of RxCIPIENTS™ FM1000, Pharmaburst, or F-Melt™, is from about 15 to 35% w/w, or from about 50% to about 90% w/w, or from about 25% to about 65% w/w, respectively.

In one embodiment, a dry pharmaceutical blend of a preferred orally disintegrating excipient, such as, for example, but not limited to, RxCIPIENTS™ FM1000, Pharmaburst, F-Melt™, and the like and low-dose doxepin, or a low-dose doxepin-related compound, can be used to produce the final dosage form by direct compression. Typically, the dry blend can contain from about 0.1% to about 10% w/w, or from about 0.5% to about 5% w/w, or from about 0.7% to about 4.5% w/w of low-dose doxepin or a low-dose doxepin-related compound. In one embodiment, the doxepin or doxepin-related compound, in the dry blend can be non-granulated. In addition to doxepin, the blend can contain from about 15% to about 30% w/w RxCIPIENTS™, or from about 25% to about 65% w/w F-Melt™, or from about 50% to about 90% w/w Pharmaburst, or the like.

In some embodiments, an orally disintegrating excipient, such as RxCIPIENTS™ FM1000, Pharmaburst, F-Melt™, and the like can be combined or replaced with one or more of the following excipients: microcrystalline cellulose, lactose monohydrate (spray dried), a compressible sugar, xylitol (Xylitab), sorbitol, mannitol, pregelatinized starch, maltodextrin, calcium phosphate dibasic, calcium phosphate tribasic, calcium carbonate DC, and the like. Accordingly, in one embodiment, one or more of the above excipients can be combined with a preferred excipient, such as RxCIPIENTS™ FM1000, Pharmaburst, or F-Melt™, and the like, in various ratios. For example, assuming the total filler to be 100%, about 80% a preferred excipient, such as RxCIPIENTS™ FM1000, Pharmaburst, or F-Melt™, can be combined with about 20% of one or more alternate filler(s). Alternatively, about 70% of a preferred excipient, such as RxCIPIENTS™ FM1000, Pharmaburst, or F-Melt™, can be combined with about 30% of one or more alternate filler(s), or about 60% of a preferred excipient, such as RxCIPIENTS™ FM1000, or Pharmaburst, F-Melt™, can be combined with about 40% of one or more alternate filler(s), or about 50% of a preferred excipient, such as RxCIPIENTS™ FM1000, or Pharmaburst, F-Melt™, can be combined with about 50% of one or more alternate filler(s), or about 40% of a preferred excipient, such as RxCIPIENTS™ FM1000, or Pharmaburst, F-Melt™, can be combined with about 60% of one or more alternate filler(s), or about 30% of a preferred excipient, such as RxCIPIENTS™ FM1000, Pharmaburst, or F-Melt™, can be combined with about 70% of one or more alternate filler(s), or about 20% of a preferred excipient, such as RxCIPIENTS™ FM1000, Pharmaburst, or F-Melt™, can be combined with about 80% of one or more alternate filler(s).

In alternate embodiments, of a preferred excipient, such as RxCIPIENTS™ FM1000, Pharmaburst, or F-Melt™, can be replaced with one or more alternate excipients. Preferably, alternate excipients are selected to provide favorable drug processing qualities. Other favorable quick dissolve delivery systems will be apparent to one of skill in the art.

The dry blend can also include at least one additional pharmaceutically acceptable suitable excipient. Additional excipients can include processing aids that improve the direct compression tablet-forming properties of the dry blend, and/or powder flowability. In the dry blend, excipients suitable for use in direct compression include, but are not limited to, binders, diluents, disintegrants, lubricants, fillers, carriers, and the like as set forth above.

In one embodiment, the formulation comprises a mixture of the drug substance with a preferred excipient, such as RxCIPIENTS™ FM1000, Pharmaburst, or F-Melt™, and the like, along with additional processing aides, such as, for example, magnesium stearate and colloidal silicon dioxide, and optionally, colorant(s).

In some embodiments, magnesium stearate can be added as a lubricant, for example, to improve powder flow, prevent the blend from adhering to tableting equipment and punch surfaces and provide lubrication to allow tablets to be cleanly ejected from tablet dies. Magnesium stearate can typically be added to pharmaceutical formulations at concentrations ranging from about 0.1% to about 5.0% w/w, or from about 0.25% to about 2% w/w, or from about 0.5% to about 1% w/w.

In some embodiments, color additives also can be included. The colorants can be used in amounts sufficient to distinguish dosage form strengths. Preferably, color additives approved for use in drugs (21 CFR 74) are added to the commercial formulations to differentiate tablet strengths. The use of other pharmaceutically acceptable colorants and combinations thereof are encompassed by the current invention.

Binders can be used, for example, to impart cohesive qualities to a formulation, and thus ensure that the resulting dosage form remains intact after compaction. Suitable binder materials include, but are not limited to, microcrystalline cellulose, gelatin, sugars (including, for example, sucrose, glucose, dextrose and maltodextrin), polyethylene glycol, waxes, natural and synthetic gums, polyvinylpyrrolidone, cellulosic polymers (including, for example, hydroxypropyl cellulose, hydroxypropyl methylcellulose, methyl cellulose, hydroxyethyl cellulose, and the like).

Accordingly, in some embodiments, the formulations disclosed herein can include at least one binder to enhance the compressibility of the major excipient(s). For example, the formulation can include at least one of the following binders in the following preferred ranges: from about 2 to about 6% w/w hydroxypropyl cellulose (Klucel), from about 2 to about 5% w/w polyvinylpyrrolidone (PVP), from about 1 to about 5% w/w methycellulose, from about 2 to about 5% hydroxypropyl methycellulose, from about 1 to about 5% w/w ethylcellulose, from about 1 to about 5% w/w sodium carboxy methylcellulose, and the like. The above ranges are exemplary preferred ranges. One of ordinary skill in the art would recognize additional binders and/or amounts that can be used in the formulations described herein. As would be recognized by one of ordinary skill in the art, when incorporated into the formulations disclosed herein, the amounts of the major filler(s) and/or other excipients can be reduced accordingly to accommodate the amount of binder added in order to keep the overall unit weight of the tablet unchanged. In one embodiment, the binder(s) is sprayed on from solution, e.g. wet granulation, to increase binding activity.

Lubricants can be employed herein in the manufacture of certain dosage forms. For example, a lubricant will often be employed when producing tablets. In an embodiment of the invention disclosed, a lubricant can be added just before the tableting step, and can be mixed with the formulation for a minimum period of time to obtain good dispersal. In some embodiments, one or more lubricants can be used. Examples of suitable lubricants include, but are not limited to, magnesium stearate, calcium stearate, zinc stearate, stearic acid, talc, glyceryl behenate, polyethylene glycol, polyethylene oxide polymers (for example, available under the registered trademarks of Carbowax® for polyethylene glycol and Polyox® for polyethylene oxide from Dow Chemical Company, Midland, Mich.), sodium lauryl sulfate, magnesium lauryl sulfate, sodium oleate, sodium stearyl fumarate, DL-leucine, colloidal silica, and others as known in the art. Preferred lubricants are sodium stearyl fumarate, magnesium stearate, calcium stearate, zinc stearate and mixtures of magnesium stearate with sodium lauryl sulfate. Lubricants can comprise from about 0.25% to about 10% of the tablet weight, more preferably from about 0.5% to about 3%.

Thus, in some embodiments, the formulations disclosed herein can include at least one lubricant in the following preferred ranges: from about 0.25 to about 2% w/w magnesium stearate, from about 0.25 to about 2% w/w calcium stearate, from about 0.25 to about 2% w/w sodium stearyl fumarate, from about 0.25 to about 2% w/w stearic acid, from about 0.25 to about 2% w/w hydrogenated vegetable oil, from about 0.25 to about 2% w/w glyceryl behenate, from about 0.25 to about 2% w/w polyethylene glycol 4000-6000, and the like. The above ranges are examples of preferred ranges. One of ordinary skill in the art would recognize additional lubricants and/or amounts that can be used in the formulations described herein. As would be recognized by one of ordinary skill in the art, when incorporated into the formulations disclosed herein, the amounts of the major filler(s) and/or other excipients can be reduced accordingly to accommodate the amount of lubricant(s) added in order to keep the overall unit weight of the tablet unchanged.

Disintegrants can be used, for example, to facilitate tablet disintegration after administration, and are generally starches, clays, celluloses, algins, gums or crosslinked polymers. Suitable disintegrants include, but are not limited to, crosslinked polyvinylpyrrolidone (PVP-XL), sodium starch glycolate, and croscarmellose sodium. If desired, the pharmaceutical formulation can also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, sodium lauryl sulfate, dioctyl sodium sulfosuccinate, polyoxyethylene sorbitan fatty acid esters, etc.

In some embodiments, at least one additional disintegrant can be included in the following preferred ranges: from about 1 to about 3% w/w croscarmellose sodium, from about 4 to about 6% w/w sodium starch glycolate, from about 2 to about 4% w/w crospovidone, from about 10 to about 20% w/w microcrystalline cellulose, from about 5 to about 10% w/w pregelatinized starch, from about 5 to about 10% w/w corn starch, from about 5 to about 10% w/w alginic acid, from about 1 to about 5% w/w ion exchange resin (Amberlite 88), and the like. The above ranges are examples of preferred ranges. One of ordinary skill in the art would recognize additional disintegrants and/or amounts of disintegrants that can be used in the formulations described herein. As would be recognized by one of ordinary skill in the art, when incorporated into the formulations disclosed herein, the amounts of the major filler(s) and/or other excipients can be reduced accordingly to accommodate the amount of disintegrant added in order to keep the overall unit weight of the tablet unchanged.

### Dosage

The selected dosage level can depend upon, for example, the route of administration, the severity of the condition being treated, and the condition and prior medical history of the patient being treated. However, it is within the skill of the art to start doses of the compound at levels lower than required to achieve the desired therapeutic effect and to gradually increase the dosage until the desired effect is achieved with an acceptable safety profile. It will be understood, however, that the specific dose level for any particular patient can depend upon a variety of factors including, for example, the genetic makeup, body weight, general health, diet, time and route of administration, combination with other drugs and the particular condition being treated, and its severity. For the treatment of insomnia, preferably one dose is administered prior to bedtime.

As used herein, the term “unit dosage form,” refers to physically discrete units suitable as unitary dosages for human and animal subjects, each unit containing a predetermined quantity of doxepin calculated in an amount sufficient to produce the desired effect in association with a pharmaceutically acceptable excipient, carrier or vehicle. In some embodiments, the unit dosage form is, for example, a tablet, a lozenge, a chewing gum, a film, a capsule, a pill, a caplet, a gel cap, a pellet, a bead, or the like. In some embodiments, the unit dosage form is a tablet. In some embodiments, the amount of doxepin in a unit dosage form is about 0.5 mg to about 9 mg, or about 1 mg to about 9 mg, or about 1 mg to about 6 mg.

In some embodiments, daily dosages of low dose doxepin can be about 1, 2, 3, 4, 5, 6, 7, 8, or 9 milligrams. In one embodiment, an initial daily dosage of about 1 milligram can be given. If the desired improvement in sleep is not achieved, then the dosage can be incrementally increased until the desired effect is achieved or until a maximum desired dosage is reached which can be, for example, 2 milligrams, 3 milligrams, 4 milligrams, 5 milligrams or 6 milligrams. It should be noted that other dosages of doxepin can be used in the embodiments described herein. For example, the dosage can be about 0.5 to about 10 milligrams.

The term “low dose” can refer to a daily dose range of between about 0.01 and 9 milligrams, or to even lower doses. In some embodiments the preferable dosage of doxepin can be between about 0.1 milligram and 9 milligrams. Preferably, the dosage can be about 0.1 milligrams, about 0.2 milligrams, about 0.3 milligrams, about 0.5 milligrams, about 1 milligram, about 2 milligrams, about 3 milligrams, about 4 milligrams, about 5 milligrams, 6 milligrams, about 7 milligrams, about 8 milligrams, or about 9 milligrams.

It should be noted that in some embodiments the formulations and methods described herein can be applied to any dosage of doxepin, including higher doses used to treat depression and anxiety. As one example, the formulations and methods can be applied to dosages between about 10 milligrams and 20 milligrams or higher.

### Methods of Making the Claimed Compositions

Pharmaceutical preparations for oral use can be obtained by mixing one or more solid excipients with a pharmaceutical composition as described herein, optionally grinding the resulting mixture, and processing the mixture of granules, after adding suitable auxiliaries, if desired, to obtain tablets. In one embodiment, the compositions are prepared using a dry granulation process. Alternatively, a wet granulation process can be used. In other embodiments, fluid bed granulation processing techniques are used.

One such granulation method is the “wet” granulation process, wherein dry solids (drug substance, filler, binder etc.) are blended and moistened with water or another wetting agent (e.g. an alcohol) and agglomerates or granules are built up of the moistened solids. Wet massing is continued until a desired homogenous particle size has been achieved whereupon the granulated product is dried.

In a preferred embodiment, the compositions disclosed herein are prepared using direct compression. In some embodiments of the invention disclosed herein, the use of wet granulation techniques is specifically excluded.

As used herein, “direct compression” means that the solid unit dosage form is prepared by compression of a simple mixture of the active pharmaceutical ingredient and excipients, without the active ingredient having been subjected to an intermediate granulation process in order to embed it in a larger particle and improve its fluidity properties.

In direct compression, the formulation ingredients, including the active pharmaceutical ingredient and processing aids, are incorporated into a free flowing blend. In one embodiment, the active ingredient, excipients, and other substances are blended and then compressed into tablets. Tablets are typically formed by pressure being applied to a material in a tablet press.

Advantages of direct compression over wet and dry granulation processes, include, for example, shorter processing times and cost advantages.

In one embodiment, a dry blend is used in forming low-dose doxepin or doxepin-related compound tablets through gravity-fed, direct compression tableting. By “gravity fed tableting press” it is meant that a pharmaceutical formulation is not force fed into a die, and that the flow of the pharmaceutical formulation is induced by gravity. An example of a gravity fed tableting press is the Manesty F-press.

Preferably, the doxepin hydrochloride tablet products disclosed herein are manufactured with common and simple processes including direct blending and compression using commercially available pharmaceutical equipment. These operations utilize readily available equipment, do not expose the API to moisture and heat, and are scalable. Preferably, the commercial manufacturing process produces and maintains blends and unit dosage forms with uniform potency that meet all quality characteristics. In a preferred embodiment, the manufacturing processes for all dosage strength formulations are the same.

The manufacturing process can include preparing one or more pre-blends which are combined to form a final blend and subsequently forming final unit dosage forms. The process can optionally include several techniques to facilitate formation of blends and batches of finished drug product with homogeneous distribution of drug substance and colorants including, for example: (1) de-agglomerating ingredients prior to blending; and/or (2) layering the drug substance and colorant components between additions of RxCIPIENTS™ FM1000, Pharmaburst, or F-Melt™, prior to mixing to create uniform pre-blends; and/or (3) serially diluting the drug substance and colorant pre-blends with RxCIPIENTS™ FM1000, Pharmaburst, or F-Melt™ and other formulation excipients to create uniform final blends. In addition, the process can optionally include, for example, (1) performing blend mixing time studies and assessing drug substance uniformity; and/or (2) optimizing the blend batch size with respect to the effective working capacity of the blenders.

Efficient mixing and acceptable blend and content uniformity can be difficult to obtain for low dose dosage forms. Preferably, the choice of blenders and the configuration of the storage container to tablet press powder transfer chute are selected based on optimization and maintenance of content uniformity. In addition, excipients and process parameters can be selected to optimize main compression force and tablet press speed on the physical characteristics (hardness, friability, thickness and weight) of the finished dosage form.

In addition, the process can be optimized to compensate for the tendency for fluidization segregation of drug substance. For example, fluidization segregation can be reduced by eliminating process steps during which streams of air come in contact with the powder, for example, the step in the process when the blend is discharged from a V-blender into storage containers, and/or the step in the process when powder is fed from storage containers to the tablet press feed hopper.

In a preferred embodiment, the formulation is simple and contains few functional components.

Preferably, the formulation can have excellent compression and flow properties and the tablet press can be operated at very high press speeds and this allows relatively manageable tablet press run times for even large batch sizes.

In some embodiments, the direct compression manufacturing processes disclosed herein achieve a uniform distribution of drug substance in a low dose drug product without the need for complex wet or dry granulation manufacturing techniques. In a preferred embodiment, the manufacturing process avoids costly techniques, such as those requiring large capital equipment investments, long manufacturing cycle times and associated low throughput.

In one embodiment, the manufacturing process is designed to achieve a uniform blend by using; multiple blending steps, a specific order of addition in the blenders and screening steps to facilitate effective dispersion of the drug substance and excipients. For example, a screening step can be introduced to prevent agglomerates of drug substance from being carried over to subsequent manufacturing steps.

In another embodiment, the manufacturing process is designed to maintain the uniform blend through to tableting via minimizing the transfer steps, for example, by using an in-bin blender to form the final blend and for example via use of a vented and valved transfer chute to the tablet press.

### Methods of Using Low Dose Doxepin or Metabolites

Embodiments relate to methods for improving sleep in a patient in need thereof, for example by administering low-dose doxepin, or a low-dose doxepin-related compound, in a tablet formulation as described herein. The term “administer” and its variants contemplate both self-administration (by the patient) and administration by a third party. In a preferred embodiment, the oral pharmaceutical as RxCIPIENTS™ FM1000, Pharmaburst, or F-Melt™-containing doxepin formulations described herein are administered orally.

As mentioned above and elsewhere, the methods described herein can be used to treat individuals suffering from a sleep disorder, such as insomnia. The individual can suffer from a chronic insomnia or a non-chronic insomnia. For chronic (e.g., greater than 3-4 weeks) or non-chronic insomnias, a patient may suffer from difficulties in sleep onset, sleep maintenance (interruption of sleep during the night by periods of wakefulness), sleep duration, sleep efficiency, premature early-morning awakening, or a combination thereof. Also, the insomnia may be attributable to the concurrent use of other medication, for example. The non-chronic insomnia can be, for example, a short term insomnia or a transient insomnia. The chronic or non-chronic insomnia can be a primary insomnia or an insomnia that is secondary or attributable to another condition, for example a disease such as depression or chronic fatigue syndrome. In some aspects, the patient can be one that is not suffering from an insomnia that is a component of a disease, or a patient can be treated that is otherwise healthy. As previously mentioned, the chronic or non-chronic insomnia can be a primary insomnia, that is, one that is not attributable to another mental disorder, a general medical condition, or a substance. In many cases, such conditions may be associated with a chronic insomnia and can include, but are not limited to, insomnia attributable to a diagnosable DSM-IV disorder, a disorder such as anxiety or depression, or a disturbance of the physiological sleep-wake system. In some aspects the insomnia can be non-chronic, or of short duration (e.g., less than 3-4 weeks). Examples of causes of such insomnia may be extrinsic or intrinsic and include, but are not limited to environmental sleep disorders as defined by the International Classification of Sleep Disorders (ICSD) such as inadequate sleep hygiene, altitude insomnia or adjustment sleep disorder (e.g., bereavement). Also, short-term insomnia may also be caused by disturbances such as shift-work sleep disorder.

It should be noted that in some aspects, the methods can specifically exclude one or more of any of the sleep disorders described in the previous paragraph or elsewhere herein. For example, without being limited thereto, in some aspects the methods can specifically exclude treating a chronic insomnia. As another example, without being limited thereto, in some aspects the methods can specifically exclude treating an insomnia that is attributable to a condition such as depression, anxiety or chronic fatigue.

In a preferred embodiment, the methods can include treating onset, duration, and maintenance aspects of insomnia in a patient.

The pharmaceutical tablet formulations disclosed herein have surprising efficacy, even in low doses, and also can allow a full 7 or 8 hours of sleep, or more, without significant next-day sedation. It is believed that these formulations are safe, provide rapid sleep onset, maintains sleep throughout the night for a full 7 or 8 hour sleep cycle, and allow normal activity the next day without hangover or unsafe levels of sedation.

### EXAMPLES

### Example 1: Plasma Concentrations

An estimate of the doxepin plasma concentrations associated with the initiation of sleep was obtained by combining data from multiple studies.

Briefly, in human subjects averaging about 70 kg, receiving a 3 mg capsule comprising doxepin hydrochloride, lactose monohydrate fast-flow, sodium lauryl sulfate and magnesium stearate, sleep onset occurred at about 60 minutes after dosing—significantly earlier than with a placebo dose. In a separate study, in humans averaging about 70 kg, receiving a 3 mg capsule comprising doxepin hydrochloride, lactose monohydrate fast-flow, sodium lauryl sulfate and magnesium stearate, the average plasma concentration at 60 minutes was approximately 0.1 ng/mL.

Without being limited, it is believed that the doxepin plasma concentration achieved approximately 1 hour after a 3 mg dose is sufficient for sleep initiation. The conclusion is that doxepin plasma concentrations of 0.1 ng/mL or greater were associated with initiation of sleep.

The formulations of the present disclosure, in contrast, can provide rapid rise in plasma concentrations following administration, e.g., achieving a therapeutically effective plasma concentration of doxepin following a 3 mg dose in 60 minutes or less, for example, within 50 minutes, 45 minutes, 40 minutes, 35 minutes, 30 minutes, 20 minutes, or less. Accordingly, some embodiments relate to formulations and dosage forms that result in more rapid achievement of effective plasma concentrations of doxepin leading to more rapid drug onset (e.g., sleep onset) at the dosages described herein, including, for example, dosages of 1 mg, 3 mg or 6 mg.

### Example 2: Doxepin Oral Transmucosal Absorption Simulation Study

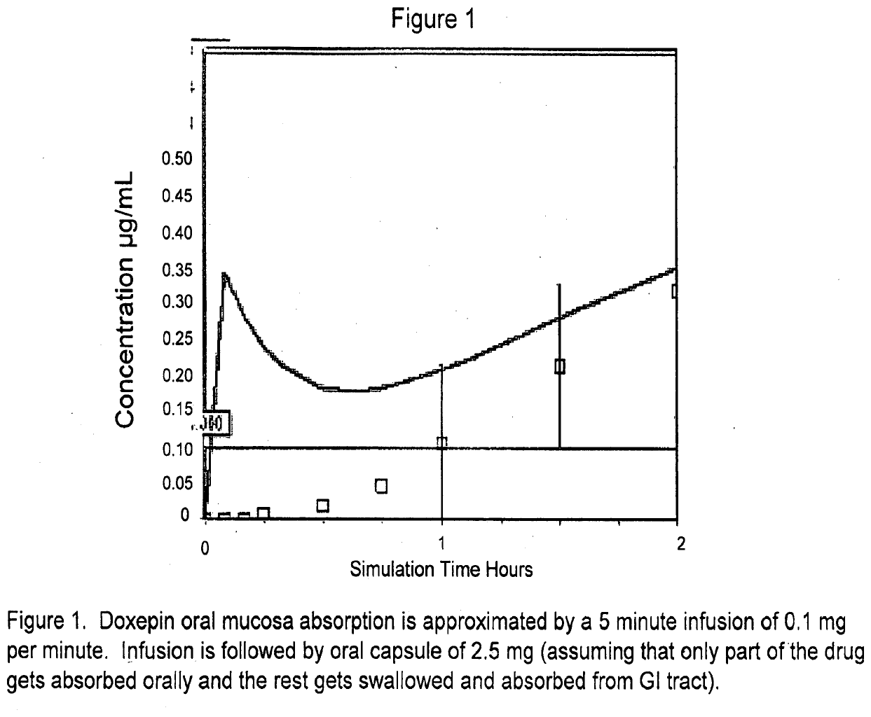

Blood plasma levels of doxepin over time from both transmucosal and GI tract absorption of a 3 mg dose of drug were simulated using the parameters shown in Table 1. Briefly, doxepin oral transmucosal absorption was approximated by a 5 minute infusion of 0.1 mg (0.5 mg total absorption). The remaining doxepin was simulated to be swallowed and absorbed in the gastrointestinal tract. FIG. 1 shows the concentration of doxepin in the blood plasma over two hours. The concentration of doxepin in the first 30 minutes of the simulation was increased above 0.1 ng/mL in the blood plasma due to oral transmucosal absorption.

TABLE 1

| | Calculated(ADMET | |
|---|---|---|
| Parameter | Predictor ™ | Measured |
| lopP | 4.27 | 4.13 |
| pKa | 8.96 | 8.96 |
| Bioavailability | Not determined | 29% |
| Plasma protein binding | 11.9% unbound | 20% unbound |
| Solubility factor | 261 | N/A |

### Example 3: 1 mg, 3 mg, and 6 mg ODT Formulations

Representative 1 mg, 3 mg, and 6 mg formulations using Pharmaburst as the quick dissolving excipient are provided in Table 2.

TABLE 2
Pharmaburst System Formulation

| Ingredient | Quantity (%) |
|---|---|
| Doxepin HCl | 4.0 |
| Pharmaburst | 82.4 |
| Crospovidone XL | 5.3 |
| Citric Acid | 1.8 |
| Flavor | 3.5 |
| Colorant | 0.6 |
| Sweetener | 0.9 |
| Sodium Stearyl Fumarate | 1.5 |

Representative 1 mg, 3 mg, and 6 mg formulations using RxCipients FM1000 as the quick dissolving excipient are provided in Table 3.

TABLE 3
RxCipients System Formulation

| Ingredient | Quantity (%) |
|---|---|
| Doxepin HCl | 4.0 |
| RxCipients FM1000 Calcium Silicate | 29.4 |
| Perlitol 200 SD (Mannitol) | 52.9 |
| Crospovidone XL | 4.9 |
| Citric Acid | 1.8 |
| Flavor | 3.5 |
| Colorant | 0.6 |
| Sweetener | 0.9 |
| Sodium Stearyl Fumarate | 1.5 |

Representative 1 mg, 3 mg, and 6 mg formulations using F-Melt as the quick dissolving excipient are provided in Table 4.

TABLE 4
F-Melt System Formulation

| Ingredient | Quantity (%) |
|---|---|
| Doxepin HCl | 3.6 |
| F-Melt (Type M) | 57.9 |
| Perlitol 200 SD (Mannitol) | 25.6 |
| Crospovidone XL | 4.9 |
| Citric Acid | 1.6 |
| Flavor | 3.5 |
| Colorant | 0.5 |
| Sweetener | 0.9 |
| Sodium Stearyl Fumarate | 1.5 |

### Example 4: Blend Uniformity

Due to the very low concentrations of drug substance in these formulations, the blending process can include preparation of a drug substance pre-blend created by layering doxepin HCl between additions of quick dissolve excipient, followed by mixing. The uniformity of unit dose potency can be further promoted by serially diluting and mixing the drug substance pre-blend with the remaining excipient(s).

Many modifications and variations of the embodiments described herein may be made without departing from the scope, as is apparent to those skilled in the art. The specific embodiments described herein are offered by way of example only.

What is claimed is:

1. A method of treating insomnia, the method comprising:
administering to a patient in need thereof a pharmaceutical formulation comprising doxepin or a pharmaceutically acceptable salt thereof and an orally disintegrating excipient, thereby providing more rapid drug onset than by administration of a similar formulation of doxepin or a pharmaceutically acceptable salt thereof without an orally disintegrating excipient.

2. The method of claim 1, wherein the pharmaceutical formulation comprises about 0.1 mg to about 9 mg of doxepin or a pharmaceutically acceptable salt thereof.

3. The method of claim 1, wherein the pharmaceutical formulation comprises about 15% to about 99.9% w/w of the orally disintegrating excipient.

4. The method of claim 1, wherein dissolution of the pharmaceutical formulation is greater than 85% within 5 minutes.

5. The method of claim 1, wherein dissolution of the pharmaceutical formulation is nearly 100% within 1 minute.

6. The method of claim 1, wherein greater than 85% of the doxepin or a pharmaceutically acceptable salt thereof is absorbed across oral mucosa within 1 minute.

7. The method of claim 1, wherein nearly 100% of the doxepin or a pharmaceutically acceptable salt thereof is absorbed across oral mucosa within 1 minute.

8. The method of claim 1, wherein the pharmaceutical formulation does not contain a pH buffer.

9. The method of claim 1, wherein the method provides a therapeutically effective plasma concentration to induce sleep.

10. The method of claim 9, wherein the therapeutically effective plasma concentration is greater than or equal to about 0.1 ng/mL.

11. The method of claim 10, wherein the therapeutically effective plasma concentration is provided within a time frame of not more than about 60 minutes.

12. The method of claim 11, wherein the therapeutically effective plasma concentration is provided within a time frame of not more than about 30 minutes.

13. A method of providing an effective plasma concentration of doxepin, the method comprising administering a pharmaceutical formulation comprising about 0.1 mg to about 9 mg of doxepin or a pharmaceutically acceptable salt thereof and an orally disintegrating excipient, wherein the effective plasma concentration is provided more rapidly than by administration of a similar formulation of doxepin or a pharmaceutically acceptable salt thereof without an orally disintegrating excipient.

14. The method of claim 13, wherein the effective plasma concentration of doxepin is sufficient to induce sleep.

15. The method of claim 14, wherein the effective plasma concentration of doxepin is greater than or equal to about 0.1 ng/mL.

16. The method of claim 13, wherein the effective plasma concentration is provided within a period of less than or equal to 60 minutes.

17. The method of claim 16, wherein the effective plasma concentration is provided within a period of less than or equal to 30 minutes.

18. The method of claim 13, wherein the pharmaceutical formulation comprises from about 1 mg to about 6 mg of doxepin or a pharmaceutically acceptable salt thereof.

19. The method of claim 13, wherein dissolution of the pharmaceutical formulation is greater than 85% within 5 minutes.

20. The method of claim 13, wherein greater than 85% of the doxepin or a pharmaceutically acceptable salt thereof is absorbed across oral mucosa within 1 minute.
